(19) United States
(12) Patent Application Publication
(10) Pub. No.: US 2019/0150439 A1
(43) Pub. Date: May 23, 2019

(54) SOLUBLE LIQUID FORMULATIONS OF QUINCLORAC AMMONIUM SALTS

(71) Applicant: BASF SE, Ludwigshafen (DE)

(72) Inventor: Wayne BARTON, Mississauga (CA)

(21) Appl. No.: 16/252,087

(22) Filed: Jan. 18, 2019

Related U.S. Application Data
(62) Division of application No. 14/390,448, filed on Oct. 3, 2014.
(60) Provisional application No. 61/620,477, filed on Apr. 5, 2012.

(30) Foreign Application Priority Data
Apr. 5, 2012 (EP) 12163304.4

(51) Int. Cl. A01N 43/42; C07D 215/02; C07C 211/62; A01N 57/20; A01N 25/02; A01N 33/12

(52) U.S. Cl. CPC A01N 43/42; C07D 215/02; A01N 33/12; A01N 57/20; A01N 25/02; C07C 211/62

(57) ABSTRACT

The present invention relates to soluble liquid (SL) formulations comprising
A) quinclorac ammonium salts of formula I;
B) a solvent of formula IIa and/or a solvent of formula IIb; and
C) at least one further herbicidal active ingredient C selected from the group consisting of c1) to c2):
   c1) enolpyruvyl shikimate 3-phosphate synthase inhibitors (EPSP inhibitors); and
   c2) glutamine synthetase inhibitors;
      including their agriculturally acceptable salts or derivatives;
      as well as to herbicidal active mixtures comprising at least one quinclorac ammonium salt of formula I and at least one further herbicide C selected from the group consisting of c1) to c2).

### CROSS REFERENCE TO RELATED APPLICATIONS

This application is a divisional of U.S. patent application Ser. No. 14/390,448, filed Oct. 3, 2014, the entire contents of which are hereby incorporated herein by reference. U.S. patent application Ser. No. 14/390,448, is a National Stage application of International Application No. PCT/EP2013/056890, filed Apr. 2, 2013, which claims the benefit of U.S. Provisional Application Ser. No. 61/620,477, filed Apr. 5, 2012, the contents of which are herein incorporated by reference in their entirety. U.S. application Ser. No. 14/390,448 also claims priority under 35 U.S.C. § 119 to European Patent Application No. 12163304.4 filed Apr. 5, 2012, the contents of which are expressly incorporated herein by reference.

The present invention relates to novel soluble liquid formulations (SL formulations) having herbicidal activity. More specifically the present invention relates to a novel herbicidally active soluble liquid formulation comprising ammonium salts of quinclorac and at least one further herbicide C in a suitable solvent.

Quinclorac (3,7-dichloro-8-quinolinecarboxylic acid) is a known herbicide. Due to its low solubility in water (0.065 mg/kg at pH 7 at 20° C.) it is usually formulated in a solid form. However, liquid formulations are sought showing an improved solubility of quinclorac and/or improved herbicidal activity.

In the case of crop protection it is desirable in principle to increase the specific activity of an active compound and the reliability of the effect. For an herbicidal composition, it is particularly desirable to control harmful plants effectively, but at the same time to be compatible with the useful plants in question. Also desirable is a broad spectrum of activity allowing the simultaneous control of harmful plants or of phytopathogenic fungi. Frequently, this cannot be achieved using a single active compound.

Thus it is one object of the present invention to provide herbicidal active mixtures and formulations having an improved efficacy against undesired weeds, preferably against small seeded broadleaf weed species, more preferably against Galium aparine.

Further, with many highly effective herbicides, there is the problem that their compatibility with useful plants, in particular dicotyledonous crop plants, such as cotton, oilseed rape and graminaceous plants, such as barley, millet, corn, rice, wheat and sugar cane, is not always satisfactory, i.e. in addition to the harmful plants, the crop plants, too, are damaged on a scale which cannot be tolerated. By reducing the application rates, the useful plants are spared; however, naturally, the extent of the control of harmful plants decreases, too.

It is known that special combinations of different specifically active herbicides result in enhanced activity of a herbicide component in the sense of a synergistic effect. In this manner, it is possible to reduce the application rates of herbicidal active compounds required for controlling the harmful plants.

Furthermore, it is known that in some cases joint application of specifically acting herbicides with other organic active compounds allows better crop plant compatibility to be achieved. In these cases, the active compounds act as antidotes or antagonists and are also referred to as safeners, since they reduce or even prevent damage to the crop plants.

Rapeseed is among the most important crop plants. Improving their growth conditions is thus an ongoing need. In the context of the present invention, the term “rapeseed” denotes both the plant itself as well as its harvested product, such rapeseed grains or seeds.

Thus it is also an object of the present invention to provide herbicidal active mixtures and formulations which are highly active against unwanted harmful plants in crops, preferably in brassica crop species, more preferably in rapeseed cultures, particularly preferred in oilseed rape and canola.

It has been found, surprisingly, that soluble liquid (SL) formulations comprising
A) quinclorac ammonium salts of formula I

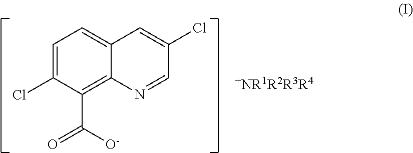

wherein
R¹, R², R³ and R⁴ are independently hydrogen, C₁-C₆-alkyl, —(CHR^{a}—CHR^{b}—Z₁)ₓ—H or —(CHR^{a}—CHR^{b}—CHR^{c}—CHR^{d}—Z₂)_{y}—H (wherein R^{a}, R^{b}, R^{c} and R^{d} are independently hydrogen or C₁-C₆-alkyl, wherein Z₁ and Z₂ are independently O, NH or N—C₁-C₆-alkyl, and wherein x and y are independently an integer from 1 to 6);
B) a solvent of formula IIa

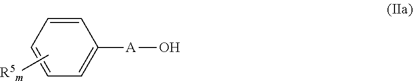

wherein
R⁵ is C₁-C₆-alkyl;
A is C₁-C₆-alkylene or C₁-C₆-oxyalkylene; and
m is 0, 1, 2 or 3;
and/or a solvent of formula IIb

HO—B—OH (IIb)
wherein
B is a straight-chain or branched C₂-C₈-alkylene or C₂-C₄-alkyleneoxy-C₂-C₄-alkylene or C₂-C₃-alkyleneoxy-C₂-C₃-alkyleneoxy-C₂-C₃-alkylene;
   and
C) at least one further herbicidal active ingredient C selected from the group consisting of c1) to c2):
   c1) enolpyruvyl shikimate 3-phosphate synthase inhibitors (EPSP inhibitors); and
   c2) glutamine synthetase inhibitors;

including their agriculturally acceptable salts or derivatives;

achieve these objectives.

The solvents of formula IIa and/or IIb provide adequate solubility of the quinclorac ammonium salts of formula I and the at least one further herbicide C.

Moreover, the use of the solvents of formula IIa and/or IIb has been found to result in very good stability of the quinclorac ammonium salts of formula I and the at least one further herbicide C in solution.

In addition the herbicidal activity of these liquid formulations is increased.

The combination of quinclorac ammonium salts of formula I with at least one further herbicidal active ingredient C provides an improved efficacy against undesired weeds, preferably against small seeded broadleaf weed species, more preferably against Galium aparine.

The present invention also relates to herbicidal active mixtures comprising at least one quinclorac ammonium salt of formula I as described herein and at least one further herbicide C selected from the group consisting of c1) to c2):
c1) enolpyruvyl shikimate 3-phosphate synthase inhibitors (EPSP inhibitors); and
c2) glutamine synthetase inhibitors;
   including their agriculturally acceptable salts or derivatives,
   as described herein.

The present invention also relates to the use of soluble liquid formulations comprising at least one quinclorac ammonium salt of formula I and at least one further herbicide C in a suitable solvent, or of compositions comprising at least one quinclorac ammonium salt of formula I and at least one further herbicide C for controlling undesired vegetation in crops, especially in rapeseed cultures.

The invention furthermore relates to a method for controlling undesired vegetation in crops, especially in rapeseed cultures, which method comprises allowing an effective amount of an SL formulation or herbicidal mixture as defined above or below to act on crop plants or parts thereof, preferably rapeseed plants or parts thereof, and/or the environment where the crops, prefrabaly the rapeseed cultures, grow or are to grow. The crop, preferably the rapeseed, might be resistant to one or more herbicides or to attack by insects owing to genetic engineering or breeding.

If the components A, B and/or C are capable of forming geometrical isomers, for example E/Z isomers, both the pure isomers and mixtures thereof may be used in the SL formulations according to the invention.

If the components A, B and/or C have one of more centers of chirality and are thus present as enantiomers or diastereomers, both the pure enantiomers and diastereomers and mixtures thereof may be used in the SL formulations according to the invention.

The preferred embodiments of the invention mentioned herein below have to be understood as being preferred either independently from each other or in combination with one another.

In a preferred embodiment of the invention the SL formulation comprises as component A) a quinclorac ammonium salt wherein
R¹ is hydrogen or C₁-C₆-alkyl;
R², R³ are independently C₁-C₆-alkyl; and
R⁴ is hydrogen.

In another preferred embodiment of the invention the SL formulation comprises as component A) a quinclorac ammonium salt wherein
R¹ is hydrogen, methyl or ethyl;
R², R³ are independently methyl or ethyl; and
R⁴ is hydrogen.

In another preferred embodiment of the invention the SL formulation comprises as component A) a quinclorac ammonium salt wherein
R¹, R⁴ are hydrogen; and
R², R³ are independently C₁-C₆-alkyl.

In another preferred embodiment of the invention the SL formulation comprises as component A) a quinclorac ammonium salt wherein
R¹, R⁴ are hydrogen; and
R², R³ are independently methyl or ethyl.

In another preferred embodiment of the invention the SL formulation comprises as component A) quinclorac dimethylammonium.

In another preferred embodiment of the invention the SL formulation comprises as component A) a quinclorac ammonium salt wherein
R¹ is hydrogen or C₁-C₆-alkyl; and
R², R³ are independently —(CHR^{a}—CHR^{b}—Z₁)ₓ-H or —(CHR^{a}—CHR^{b}—CHR^{c}—CHR^{d}—Z₂)_{y}—H (wherein R^{a}, R^{b}, R^{c} and R^{d} are independently hydrogen or C₁-C₆-alkyl, wherein Z₁ and Z₂ are independently 0, NH or N—C₁-C₆-alkyl, and wherein x and y are independently an integer from 1 to 6);
R⁴ is hydrogen.

In another preferred embodiment of the invention the SL formulation comprises as component A) a quinclorac ammonium salt wherein
R¹ is hydrogen or C₁-C₆-alkyl;
R² is —(CHR^{a}—CHR^{b}—Z₁)ₓ-H or —(CHR^{a}—CHR^{b}—CHR^{c}—CHR^{d}—Z₂)_{y}—H, (wherein R^{a}, R^{b}, R^{c} and R^{d} are independently hydrogen or C₁-C₆-alkyl, wherein Z₁ and Z₂ are independently O, NH or N—C₁-C₆-alkyl, and wherein x and y are independently an integer from 1 to 6; preferably wherein Z₁ and Z₂ are O); especially —(CH₂—CH₂—O)ₓH or —(CH₂—CH₂—CH₂—CH₂—O)_{y}—H and wherein m and n are independently an integer from 1 to 6; and
R³, R⁴ are hydrogen.

In another preferred embodiment of the invention the SL formulation comprises as component A) a quinclorac ammonium salt wherein
R¹ is hydrogen, methyl or ethyl;
R² is C₁-C₆-hydroxyalkyl or C₁-C₆-hydroxyalkoxy-C₁-C₆-alkyl; and
R³, R⁴ are hydrogen.

In another preferred embodiment of the invention the SL formulation comprises as component A) a quinclorac ammonium salt wherein
R¹, R³, R⁴ are hydrogen; and
R² is 2-hydroxyethyl or 2-(2-hydroxyethoxy)ethyl.

In another preferred embodiment of the invention the SL formulation comprises as component A) a quinclorac ammonium salt wherein
R¹, R³, R⁴ are hydrogen; and
R² is 2-(2-hydroxyethoxy)ethyl.

The SL formulation according to the present invention comprises as component B) a solvent of formula IIa and/or a solvent of formula IIb.

In a preferred embodiment of the invention the SL formulation comprises as component B) only a solvent of formula IIa.

In another preferred embodiment of the invention the SL formulation comprises as component B) only a solvent of formula IIa, wherein
m is 0; and
   R⁵ and A have the meanings as given above.

In another preferred embodiment of the invention the SL formulation comprises as component B) only a solvent of formula IIa, wherein
A is methylene, ethylene or oxyethylene;
m is 0; and
   R⁵ has the meaning as given above.

In another preferred embodiment of the invention the SL formulation comprises as component B) only benzyl alcohol.

In another preferred embodiment of the invention the SL formulation comprises as component B) only a solvent of formula IIb.

In another preferred embodiment of the invention the SL formulation comprises as component B) only a solvent of formula IIb wherein
B is a straight-chain or branched C₂-C₈-alkylene or C₂-C₄-alkyleneoxy-C₂-C₄-alkylene.

In another preferred embodiment of the invention the SL formulation comprises as component B) a only solvent of formula IIb wherein
B is ethylene, propylene or ethyleneoxyethylene.

In another preferred embodiment of the invention the SL formulation comprises as component B) only glycol (ethylene glycol; HO—CH₂CH₂—OH).

In another preferred embodiment of the invention the SL formulation comprises as component B) a solvent of formula IIa and a solvent of formula IIb.

In another preferred embodiment of the invention the SL formulation comprises as component B)

a solvent of formula IIa wherein
m is 0;
R⁵ and A have the meanings as given above; and
   a solvent of formula IIb wherein
B is a straight-chain or branched C₂-C₈-alkylene or C₂-C₄-alkyleneoxy-C₂-C₄-alkylene.

In another preferred embodiment of the invention the SL formulation comprises as component B)

a solvent of formula IIa wherein
A is C₁-C₆-alkylene or C₁-C₆-oxyalkylene;
m is 0;
R⁵ has the meanings as given above; and
   a solvent of formula IIb wherein
B is ethylene, propylene or ethyleneoxyethylene.

In another preferred embodiment of the invention the SL formulation comprises as component B) benzyl alcohol and glycol.

The SL formulation according to the present invention comprises in addition to components A) and B) one or more (at least one) additional herbicidal active ingredient(s) C [component(s) C; herbicide(s) C] selected from the group consisting of c1) to c2):
c1) enolpyruvyl shikimate 3-phosphate synthase inhibitors (EPSP inhibitors); and
c2) glutamine synthetase inhibitors;
   including their agriculturally acceptable salts or derivatives.

Preferred herbicides C which can be used in the SL formulation according to the present invention are:
c1) from the group of the EPSP synthase inhibitors: glyphosate;
c2) from the group of the glutamine synthase inhibitors: bilanaphos (bialaphos), glufosinate and glufosinate-P;
   including their agriculturally acceptable salts or derivatives.

If the components C have ionizable functional groups, they can also be employed in the form of their agriculturally acceptable salts. Suitable are, in general, the salts of those cations and the acid addition salts of those acids whose cations and anions, respectively, have no adverse effect on the activity of the active compounds.

Preferred cations are the ions of the alkali metals, preferably of lithium, sodium and potassium, of the alkaline earth metals, preferably of calcium and magnesium, and of the transition metals, preferably of manganese, copper, zinc and iron, further ammonium and substituted ammonium in which one to four hydrogen atoms are replaced by C₁-C₄-alkyl, hydroxy-C₁-C₄-alkyl, C₁-C₄-alkoxy-C₁-C₄-alkyl, hydroxy-C₁-C₄-alkoxy-C₁-C₄-alkyl, phenyl or benzyl, preferably ammonium, methylammonium, isopropylammonium, dimethylammonium, diisopropylammonium, trimethylammonium, heptylammonium, dodecylammonium, tetradecylammonium, tetramethylammonium, tetraethylammonium, tetrabutylammonium, 2-hydroxyethylammonium (olamine salt), 2-(2-hydroxyeth-1-oxy)eth-1-ylammonium (diglycolamine salt), di(2-hydroxyeth-1-yl)ammonium (diolamine salt), tris(2-hydroxyethyl)ammonium (trolamine salt), tris(2-hydroxypropyl)ammonium, benzyltrimethylammonium, benzyltriethylammonium, N,N,N-trimethylethanolammonium (choline salt), furthermore phosphonium ions, sulfonium ions, preferably tri(C₁-C₄-alkyl)sulfonium, such as trimethylsulfonium, and sulfoxonium ions, preferably tri(C₁-C₄-alkyl)sulfoxonium, and finally the salts of polybasic amines such as N,N-bis-(3-aminopropyl)methylamine and diethylenetriamine.

Anions of useful acid addition salts are primarily chloride, bromide, fluoride, iodide, hydrogensulfate, methylsulfate, sulfate, dihydrogenphosphate, hydrogenphosphate, nitrate, bicarbonate, carbonate, hexafluorosilicate, hexafluorophosphate, benzoate and also the anions of C₁-C₄-alkanoic acids, preferably formate, acetate, propionate and butyrate.

Active components C having a carboxyl group can be employed in the form of the acid, in the form of an agriculturally suitable salt as mentioned above or else in the form of an agriculturally acceptable derivative in the compositions according to the invention, for example as amides, such as mono- and di-C₁-C₆-alkylamides or arylamides, as esters, for example as allyl esters, propargyl esters, C₁-C₁₀-alkyl esters, alkoxyalkyl esters, tefuryl ((tetrahydrofuran-2-yl)methyl) esters and also as thioesters, for example as C₁-C₁₀-alkylthio esters. Preferred mono- and di-C₁-C₆-alkylamides are the methyl and the dimethylamides. Preferred arylamides are, for example, the anilides and the 2-chloroanilides. Preferred alkyl esters are, for example, the methyl, ethyl, propyl, isopropyl, butyl, isobutyl, pentyl, mexyl (1-methylhexyl), meptyl (1-methylheptyl), heptyl, octyl or isooctyl (2-ethylhexyl) esters. Preferred C₁-C₄-alkoxy-C₁-C₄-alkyl esters are the straight-chain or branched C₁-C₄-alkoxy ethyl esters, for example the 2-methoxyethyl, 2-ethoxyethyl, 2-butoxyethyl (butotyl), 2-butoxypropyl or 3-butoxypropyl ester. An example of a straight-chain or branched C₁-C₁₀-alkylthio ester is the ethylthio ester.

Suitable salts of glyphosate are for example glyphosate-ammonium, glyphosate-diammonium, glyphoste-dimethylammonium, glyphosate-isopropylammonium, glyphosatepotassium, glyphosate-sodium, glyphosate-trimesium as well as the ethanolamine and diethanolamine salts, preferably glyphosate-diammonium, glyphosate-isopropylammonium and glyphosate-trimesium (sulfosate).

A suitable salt of glufosinate is for example glufosinate-ammonium.

A suitable salt of glufosinate-P is for example glufosinate-P-ammonium.

Particularly preferred herbicides C (components C) which can be used in the SL formulation according to the present invention are selected from the group consisting of
c1) EPSP synthase inhibitors: glyphosate, glyphosate-isopropylammonium, glyposate-potassium and glyphosate-trimesium (sulfosate); and
c2) glutamine synthase inhibitors: bilanaphos (bialaphos), bilanaphos-sodium, glufosinate, glufosinate-P, glufosinate-ammonium and glufosinate-P-ammonium;
   especially preferred selected from the group consisting of
c1) EPSP synthase inhibitors: glyphosate, glyphosate-isopropylammonium and glyphosate-trimesium (sulfosate); and
c2) glutamine synthase inhibitors: glufosinate, glufosinate-P, glufosinate-ammonium and glufosinate-P-ammonium;
   more preferred selected from the group consisting of glyphosate-isopropylammonium, glyphosate-trimesium (sulfosate), glufosinate-ammonium and glufosinate-P-ammonium;
   most preferred selected from the group consisting of glyphosate-isopropylammonium and glufosinate-ammonium.

According to a specific embodiment of the invention, the SL formulation comprises in addition to components A) and B) at least one, preferably exactly one component C selected from the group of c1): EPSP synthase inhibitors including their agriculturally acceptable salts or derivatives;
preferably selected from glyphosate including its agriculturally acceptable salts or derivatives;
particularly preferred glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium.

According to another specific embodiment of the invention, the SL formulation comprises in addition to components A) and B) at least one, preferably exactly one component C selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium.

According to another specific embodiment of the invention, the SL formulation comprises in addition to components A) and B) at least two, preferably exactly two components C different from each other, wherein at least one component C is selected from the group of c1): EPSP synthase inhibitors including their agriculturally acceptable salts or derivatives;
preferably selected from glyphosate including its agriculturally acceptable salts or derivatives;
particularly preferred glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium.

According to another specific embodiment of the invention, the SL formulation comprises in addition to components A) and B) at least two, preferably exactly two components C different from each other, wherein at least one component C is selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium.

According to another specific embodiment of the invention, the SL formulation comprises in addition to components A) and B) at least two, preferably exactly two components C different from each other, wherein the first of the two or more, preferably exactly two, components C is selected from glyphosate including its agriculturally acceptable salts or derivatives;
particularly preferred glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium.
and the second of the two or more, preferably exactly two, components C is selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium.

In another preferred embodiment of the invention the SL formulation comprises as component A) a quinclorac ammonium salt wherein
R¹, R⁴ are hydrogen; and
R², R³ are independently C₁-C₆-alkyl;
   as component B) only a solvent of formula IIa wherein
A is C₁-C₆-alkylene or C₁-C₆-oxyalkylene; and
m is 0; and
   as component C) at least one herbicide selected from the group consisting of
c1) EPSP synthase inhibitors: glyphosate; and
c2) glutamine synthase inhibitors: bilanaphos (bialaphos), glufosinate and glufosinate-P;

including their agriculturally acceptable salts or derivatives;

particularly preferred selected from the group consisting of
c1) EPSP synthase inhibitors: glyphosate, glyphosate-isopropylammonium, glyposate-potassium and glyphosate-trimesium (sulfosate); and
c2) glutamine synthase inhibitors: bilanaphos (bialaphos), bilanaphos-sodium, glufosinate, glufosinate-P, glufosinate-ammonium and glufosinate-P-ammonium;
   especially preferred selected from the group consisting of
c1) EPSP synthase inhibitors: glyphosate, glyphosate-isopropylammonium and glyphosate-trimesium (sulfosate); and
c2) glutamine synthase inhibitors: glufosinate, glufosinate-P, glufosinate-ammonium and glufosinate-P-ammonium;
   more preferred selected from the group consisting of glyphosate-isopropylammonium, glyphosate-trimesium (sulfosate), glufosinate-ammonium and glufosinate-P-ammonium;
   most preferred selected from the group consisting of glyphosate-isopropylammonium and glufosinate-ammonium.

In another preferred embodiment of the invention the SL formulation comprises as component A) a quinclorac ammonium salt wherein
R¹, R⁴ are hydrogen; and
R², R³ are independently C₁-C₆-alkyl;
   as component B) only a solvent of formula IIa wherein
A is methylene, ethylene or oxyethylene; and
m is 0; and
   as component C) at least one herbicide selected from the group consisting of
c1) EPSP synthase inhibitors: glyphosate; and
c2) glutamine synthase inhibitors: bilanaphos (bialaphos), glufosinate and glufosinate-P;

including their agriculturally acceptable salts or derivatives;

particularly preferred selected from the group consisting of
c1) EPSP synthase inhibitors: glyphosate, glyphosate-isopropylammonium, glyposate-potassium and glyphosate-trimesium (sulfosate); and
c2) glutamine synthase inhibitors: bilanaphos (bialaphos), bilanaphos-sodium, glufosinate, glufosinate-P, glufosinate-ammonium and glufosinate-P-ammonium;
   especially preferred selected from the group consisting of
c1) EPSP synthase inhibitors: glyphosate, glyphosate-isopropylammonium and glyphosate-trimesium (sulfosate); and
c2) glutamine synthase inhibitors: glufosinate, glufosinate-P, glufosinate-ammonium and glufosinate-P-ammonium;
   more preferred selected from the group consisting of glyphosate-isopropylammonium, glyphosate-trimesium (sulfosate), glufosinate-ammonium and glufosinate-P-ammonium;
   most preferred selected from the group consisting of glyphosate-isopropylammonium and glufosinate-ammonium.

In another preferred embodiment of the invention the SL formulation comprises as component A) a quinclorac ammonium salt wherein
R¹, R³, R⁴ are hydrogen;
R² is 2-hydroxyethyl or 2-(2-hydroxyethoxy)ethyl;
   as component B) only a solvent of formula IIa wherein
A is C₁-C₆-alkylene or C₁-C₆-oxyalkylene;
m is 0; and
   as component C) at least one herbicide selected from the group consisting of
c1) EPSP synthase inhibitors: glyphosate; and
c2) glutamine synthase inhibitors: bilanaphos (bialaphos), glufosinate and glufosinate-P;
including their agriculturally acceptable salts or derivatives;
   particularly preferred selected from the group consisting of
c1) EPSP synthase inhibitors: glyphosate, glyphosate-isopropylammonium, glyposate-potassium and glyphosate-trimesium (sulfosate); and
c2) glutamine synthase inhibitors: bilanaphos (bialaphos), bilanaphos-sodium, glufosinate, glufosinate-P, glufosinate-ammonium and glufosinate-P-ammonium;
   especially preferred selected from the group consisting of
c1) EPSP synthase inhibitors: glyphosate, glyphosate-isopropylammonium and glyphosate-trimesium (sulfosate); and
c2) glutamine synthase inhibitors: glufosinate, glufosinate-P, glufosinate-ammonium and glufosinate-P-ammonium;
   more preferred selected from the group consisting of glyphosate-isopropylammonium, glyphosate-trimesium (sulfosate), glufosinate-ammonium and glufosinate-P-ammonium;
   most preferred selected from the group consisting of glyphosate-isopropylammonium and glufosinate-ammonium.

In another preferred embodiment of the invention the SL formulation comprises as component A) a quinclorac ammonium salt wherein
R¹, R³, R⁴ are hydrogen;
R² is 2-hydroxyethyl or 2-(2-hydroxyethoxy)ethyl;
   as component B) only a solvent of formula IIa wherein
A is methylene, ethylene or oxyethylene;
m is 0; and
   as component C) at least one herbicide selected from the group consisting of
c1) EPSP synthase inhibitors: glyphosate; and
c2) glutamine synthase inhibitors: bilanaphos (bialaphos), glufosinate and glufosinate-P;

including their agriculturally acceptable salts or derivatives;

particularly preferred selected from the group consisting of
c1) EPSP synthase inhibitors: glyphosate, glyphosate-isopropylammonium, glyposate-potassium and glyphosate-trimesium (sulfosate); and
c2) glutamine synthase inhibitors: bilanaphos (bialaphos), bilanaphos-sodium, glufosinate, glufosinate-P, glufosinate-ammonium and glufosinate-P-ammonium;

especially preferred selected from the group consisting of
c1) EPSP synthase inhibitors: glyphosate, glyphosate-isopropylammonium and glyphosate-trimesium (sulfosate); and
c2) glutamine synthase inhibitors: glufosinate, glufosinate-P, glufosinate-ammonium and glufosinate-P-ammonium;
   more preferred selected from the group consisting of glyphosate-isopropylammonium, glyphosate-trimesium (sulfosate), glufosinate-ammonium and glufosinate-P-ammonium;
   most preferred selected from the group consisting of glyphosate-isopropylammonium and glufosinate-ammonium.

In another preferred embodiment of the invention the SL formulation comprises as component A) a quinclorac ammonium salt wherein
R¹, R⁴ are hydrogen;
R², R³ are independently C₁-C₆-alkyl;
   as component B) only a solvent of formula IIb wherein
B is a straight-chain or branched C₂-C₈-alkylene or C₂-C₄-alkyleneoxy-C₂-C₄-alkylene; and
   as component C) at least one herbicide selected from the group consisting of
c1) EPSP synthase inhibitors: glyphosate; and
c2) glutamine synthase inhibitors: bilanaphos (bialaphos), glufosinate and glufosinate-P;

including their agriculturally acceptable salts or derivatives;

particularly preferred selected from the group consisting of
c1) EPSP synthase inhibitors: glyphosate, glyphosate-isopropylammonium, glyposate-potassium and glyphosate-trimesium (sulfosate); and
c2) glutamine synthase inhibitors: bilanaphos (bialaphos), bilanaphos-sodium, glufosinate, glufosinate-P, glufosinate-ammonium and glufosinate-P-ammonium;
   especially preferred selected from the group consisting of
c1) EPSP synthase inhibitors: glyphosate, glyphosate-isopropylammonium and glyphosate-trimesium (sulfosate); and
c2) glutamine synthase inhibitors: glufosinate, glufosinate-P, glufosinate-ammonium and glufosinate-P-ammonium;
   more preferred selected from the group consisting of glyphosate-isopropylammonium, glyphosate-trimesium (sulfosate), glufosinate-ammonium and glufosinate-P-ammonium;
   most preferred selected from the group consisting of glyphosate-isopropylammonium and glufosinate-ammonium.

In another preferred embodiment of the invention the SL formulation comprises as component A) a quinclorac ammonium salt wherein
R¹, R⁴ are hydrogen;
R², R³ are independently C₁-C₆-alkyl;
as component B) only a solvent of formula IIb wherein
B is ethylene, propylene or ethyleneoxyethylene; and
   as component C) at least one herbicide selected from the group consisting of
c1) EPSP synthase inhibitors: glyphosate; and
c2) glutamine synthase inhibitors: bilanaphos (bialaphos), glufosinate and glufosinate-P;

including their agriculturally acceptable salts or derivatives;

particularly preferred selected from the group consisting of
c1) EPSP synthase inhibitors: glyphosate, glyphosate-isopropylammonium, glyposate-potassium and glyphosate-trimesium (sulfosate); and
c2) glutamine synthase inhibitors: bilanaphos (bialaphos), bilanaphos-sodium, glufosinate, glufosinate-P, glufosinate-ammonium and glufosinate-P-ammonium;
   especially preferred selected from the group consisting of
c1) EPSP synthase inhibitors: glyphosate, glyphosate-isopropylammonium and glyphosate-trimesium (sulfosate); and
c2) glutamine synthase inhibitors: glufosinate, glufosinate-P, glufosinate-ammonium and glufosinate-P-ammonium;
   more preferred selected from the group consisting of glyphosate-isopropylammonium, glyphosate-trimesium (sulfosate), glufosinate-ammonium and glufosinate-P-ammonium;
most preferred selected from the group consisting of glyphosate-isopropylammonium and glufosinate-ammonium.

In another preferred embodiment of the invention the SL formulation comprises as component A) a quinclorac ammonium salt wherein
R¹, R³, R⁴ are hydrogen;
R² is 2-hydroxyethyl or 2-(2-hydroxyethoxy)ethyl;
   as component B) only a solvent of formula IIb wherein
B is a straight-chain or branched C₂-C₈-alkylene or C₂-C₄-alkyleneoxy-C₂-C₄-alkylene; and
   as component C) at least one herbicide selected from the group consisting of
c1) EPSP synthase inhibitors: glyphosate; and
c2) glutamine synthase inhibitors: bilanaphos (bialaphos), glufosinate and glufosinate-P;

including their agriculturally acceptable salts or derivatives;

particularly preferred selected from the group consisting of
c1) EPSP synthase inhibitors: glyphosate, glyphosate-isopropylammonium, glyposate-potassium and glyphosate-trimesium (sulfosate); and
c2) glutamine synthase inhibitors: bilanaphos (bialaphos), bilanaphos-sodium, glufosinate, glufosinate-P, glufosinate-ammonium and glufosinate-P-ammonium;
   especially preferred selected from the group consisting of
c1) EPSP synthase inhibitors: glyphosate, glyphosate-isopropylammonium and glyphosate-trimesium (sulfosate); and
c2) glutamine synthase inhibitors: glufosinate, glufosinate-P, glufosinate-ammonium and glufosinate-P-ammonium;
   more preferred selected from the group consisting of glyphosate-isopropylammonium, glyphosate-trimesium (sulfosate), glufosinate-ammonium and glufosinate-P-ammonium;
   most preferred selected from the group consisting of glyphosate-isopropylammonium and glufosinate-ammonium.

In another preferred embodiment of the invention the SL formulation comprises as component A) a quinclorac ammonium salt wherein
R¹, R³, R⁴ are hydrogen;
R² is 2-hydroxyethyl or 2-(2-hydroxyethoxy)ethyl;
   as component B) only a solvent of formula IIb wherein
B is ethylene, propylene or ethyleneoxyethylene; and as component C) at least one herbicide selected from the group consisting of
   c1) EPSP synthase inhibitors: glyphosate; and
   c2) glutamine synthase inhibitors: bilanaphos (bialaphos), glufosinate and glufosinate-P;

including their agriculturally acceptable salts or derivatives;

particularly preferred selected from the group consisting of
c1) EPSP synthase inhibitors: glyphosate, glyphosate-isopropylammonium, glyposate-potassium and glyphosate-trimesium (sulfosate); and
c2) glutamine synthase inhibitors: bilanaphos (bialaphos), bilanaphos-sodium, glufosinate, glufosinate-P, glufosinate-ammonium and glufosinate-P-ammonium;
   especially preferred selected from the group consisting of
c1) EPSP synthase inhibitors: glyphosate, glyphosate-isopropylammonium and glyphosate-trimesium (sulfosate); and
c2) glutamine synthase inhibitors: glufosinate, glufosinate-P, glufosinate-ammonium and glufosinate-P-ammonium;
   more preferred selected from the group consisting of glyphosate-isopropylammonium, glyphosate-trimesium (sulfosate), glufosinate-ammonium and glufosinate-P-ammonium;

most preferred selected from the group consisting of glyphosate-isopropylammonium and glufosinate-ammonium.

In another preferred embodiment of the invention the SL formulation comprises as component A) a quinclorac ammonium salt wherein
R¹, R⁴ are hydrogen;
R², R³ are independently C₁-C₆-alkyl;
   as component B) benzyl alcohol and glycol; and
   as component C) at least one herbicide selected from the group consisting of
c1) EPSP synthase inhibitors: glyphosate; and
c2) glutamine synthase inhibitors: bilanaphos (bialaphos), glufosinate and glufosinate-P;

including their agriculturally acceptable salts or derivatives;

particularly preferred selected from the group consisting of
c1) EPSP synthase inhibitors: glyphosate, glyphosate-isopropylammonium, glyposate-potassium and glyphosate-trimesium (sulfosate); and
c2) glutamine synthase inhibitors: bilanaphos (bialaphos), bilanaphos-sodium, glufosinate, glufosinate-P, glufosinate-ammonium and glufosinate-P-ammonium;
   especially preferred selected from the group consisting of
c1) EPSP synthase inhibitors: glyphosate, glyphosate-isopropylammonium and glyphosate-trimesium (sulfosate); and
c2) glutamine synthase inhibitors: glufosinate, glufosinate-P, glufosinate-ammonium and glufosinate-P-ammonium;
   more preferred selected from the group consisting of glyphosate-isopropylammonium, glyphosate-trimesium (sulfosate), glufosinate-ammonium and glufosinate-P-ammonium;
   most preferred selected from the group consisting of glyphosate-isopropylammonium and glufosinate-ammonium.

In another preferred embodiment of the invention the SL formulation comprises as component A) a quinclorac ammonium salt wherein
R¹, R³, R⁴ are hydrogen;
R² is 2-hydroxyethyl or 2-(2-hydroxyethoxy)ethyl;
   as component B) benzyl alcohol and glycol; and
   as component C) at least one herbicide selected from the group consisting of
c1) EPSP synthase inhibitors: glyphosate; and
c2) glutamine synthase inhibitors: bilanaphos (bialaphos), glufosinate and glufosinate-P;

including their agriculturally acceptable salts or derivatives;

particularly preferred selected from the group consisting of
c1) EPSP synthase inhibitors: glyphosate, glyphosate-isopropylammonium, glyposate-potassium and glyphosate-trimesium (sulfosate); and
c2) glutamine synthase inhibitors: bilanaphos (bialaphos), bilanaphos-sodium, glufosinate, glufosinate-P, glufosinate-ammonium and glufosinate-P-ammonium;
   especially preferred selected from the group consisting of
c1) EPSP synthase inhibitors: glyphosate, glyphosate-isopropylammonium and glyphosate-trimesium (sulfosate); and
c2) glutamine synthase inhibitors: glufosinate, glufosinate-P, glufosinate-ammonium and glufosinate-P-ammonium;
   more preferred selected from the group consisting of glyphosate-isopropylammonium, glyphosate-trimesium (sulfosate), glufosinate-ammonium and glufosinate-P-ammonium;
   most preferred selected from the group consisting of glyphosate-isopropylammonium and glufosinate-ammonium.

In another preferred embodiment of the invention the SL formulation comprises as component A) quinclorac dimethylammonium, as component B) benzyl alcohol and as component C) at least one herbicide selected from the group consisting of
c1) EPSP synthase inhibitors: glyphosate; and
c2) glutamine synthase inhibitors: bilanaphos (bialaphos), glufosinate and glufosinate-P;

including their agriculturally acceptable salts or derivatives;

particularly preferred selected from the group consisting of
c1) EPSP synthase inhibitors: glyphosate, glyphosate-isopropylammonium, glyposate-potassium and glyphosate-trimesium (sulfosate); and
c2) glutamine synthase inhibitors: bilanaphos (bialaphos), bilanaphos-sodium, glufosinate, glufosinate-P, glufosinate-ammonium and glufosinate-P-ammonium;
   especially preferred selected from the group consisting of
c1) EPSP synthase inhibitors: glyphosate, glyphosate-isopropylammonium and glyphosate-trimesium (sulfosate); and
c2) glutamine synthase inhibitors: glufosinate, glufosinate-P, glufosinate-ammonium and glufosinate-P-ammonium;
   more preferred selected from the group consisting of glyphosate-isopropylammonium, glyphosate-trimesium (sulfosate), glufosinate-ammonium and glufosinate-P-ammonium;
   most preferred selected from the group consisting of glyphosate-isopropylammonium and glufosinate-ammonium.

In another preferred embodiment of the invention the SL formulation comprises as component A) quinclorac dimethylammonium, as component B) benzyl alcohol and at least one, preferably exactly one component C selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium.

In another preferred embodiment of the invention the SL formulation comprises as component A) quinclorac dimethylammonium, as component B) benzyl alcohol and at least one, preferably exactly one component C selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium.

In another preferred embodiment of the invention the SL formulation comprises as component A) quinclorac dimethylammonium, as component B) benzyl alcohol and at least two, preferably exactly two, components C different from each other, wherein at least one component C is selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium.

In another preferred embodiment of the invention the SL formulation comprises as component A) quinclorac dimethylammonium, as component B) benzyl alcohol and at least two, preferably exactly two, components C different from each other, wherein at least one component C is selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium.

In another preferred embodiment of the invention the SL formulation comprises as component A) quinclorac dimethylammonium, as component B) benzyl alcohol and at least two, preferably exactly two, components C different from each other, wherein the first of the two or more, preferably exactly two, components C is selected from glyphosate including its agriculturally acceptable salts or derivatives;
particularly preferred glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium; and
   the second of the two or more, preferably exactly two, components C is selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium.

In another preferred embodiment of the invention the SL formulation comprises as component A) quinclorac dimethylammonium, as component B) glycol and as component C) at least one herbicide selected from the group consisting of
c1) EPSP synthase inhibitors: glyphosate; and
c2) glutamine synthase inhibitors: bilanaphos (bialaphos), glufosinate and glufosinate-P;

including their agriculturally acceptable salts or derivatives;

particularly preferred selected from the group consisting of
c1) EPSP synthase inhibitors: glyphosate, glyphosate-isopropylammonium, glyposate-potassium and glyphosate-trimesium (sulfosate); and
c2) glutamine synthase inhibitors: bilanaphos (bialaphos), bilanaphos-sodium, glufosinate, glufosinate-P, glufosinate-ammonium and glufosinate-P-ammonium;
   especially preferred selected from the group consisting of
c1) EPSP synthase inhibitors: glyphosate, glyphosate-isopropylammonium and glyphosate-trimesium (sulfosate); and
c2) glutamine synthase inhibitors: glufosinate, glufosinate-P, glufosinate-ammonium and glufosinate-P-ammonium;
   more preferred selected from the group consisting of glyphosate-isopropylammonium, glyphosate-trimesium (sulfosate), glufosinate-ammonium and glufosinate-P-ammonium;
   most preferred selected from the group consisting of glyphosate-isopropylammonium and glufosinate-ammonium.

In another preferred embodiment of the invention the SL formulation comprises as component A) quinclorac dimethylammonium, as component B) glycol and at least one, preferably exactly one component C selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium.

In another preferred embodiment of the invention the SL formulation comprises as component A) quinclorac dimethylammonium, as component B) glycol and at least one, preferably exactly one component C selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium.

In another preferred embodiment of the invention the SL formulation comprises as component A) quinclorac dimethylammonium, as component B) glycol and at least two, preferably exactly two, components C different from each other, wherein at least one component C is selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium.

In another preferred embodiment of the invention the SL formulation comprises as component A) quinclorac dimethylammonium, as component B) glycol and at least two, preferably exactly two, components C different from each other, wherein at least one component C is selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium.

In another preferred embodiment of the invention the SL formulation comprises as component A) quinclorac dimethylammonium, as component B) glycol and at least two, preferably exactly two, components C different from each other, wherein

the first of the two or more, preferably exactly two, components C is selected from glyphosate including its agriculturally acceptable salts or derivatives;
particularly preferred glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium; and
   the second of the two or more, preferably exactly two, components C is selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium.

In another preferred embodiment of the invention the SL formulation comprises as component A) quinclorac dimethylammonium, as component B) benzyl alcohol and glycol and as component C) at least one herbicide selected from the group consisting of
c1) EPSP synthase inhibitors: glyphosate; and
c2) glutamine synthase inhibitors: bilanaphos (bialaphos), glufosinate and glufosinate-P;

including their agriculturally acceptable salts or derivatives;

particularly preferred selected from the group consisting of
c1) EPSP synthase inhibitors: glyphosate, glyphosate-isopropylammonium, glyposate-potassium and glyphosate-trimesium (sulfosate); and
c2) glutamine synthase inhibitors: bilanaphos (bialaphos), bilanaphos-sodium, glufosinate, glufosinate-P, glufosinate-ammonium and glufosinate-P-ammonium;
   especially preferred selected from the group consisting of
c1) EPSP synthase inhibitors: glyphosate, glyphosate-isopropylammonium and glyphosate-trimesium (sulfosate); and
c2) glutamine synthase inhibitors: glufosinate, glufosinate-P, glufosinate-ammonium and glufosinate-P-ammonium;
   more preferred selected from the group consisting of glyphosate-isopropylammonium, glyphosate-trimesium (sulfosate), glufosinate-ammonium and glufosinate-P-ammonium;
   most preferred selected from the group consisting of glyphosate-isopropylammonium and glufosinate-ammonium.

In another preferred embodiment of the invention the SL formulation comprises as component A) quinclorac dimethylammonium, as component B) benzyl alcohol and glycol and at least one, preferably exactly one component C selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium.

In another preferred embodiment of the invention the SL formulation comprises as component A) quinclorac dimethylammonium, as component B) benzyl alcohol and glycol and at least one, preferably exactly one component C selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium.

In another preferred embodiment of the invention the SL formulation comprises as component A) quinclorac dimethylammonium, as component B) benzyl alcohol and glycol and at least two, preferably exactly two, components C different from each other, wherein at least one component C is selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium.

In another preferred embodiment of the invention the SL formulation comprises as component A) quinclorac dimethylammonium, as component B) benzyl alcohol and glycol and at least two, preferably exactly two, components C different from each other, wherein at least one component C is selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium.

In another preferred embodiment of the invention the SL formulation comprises as component A) quinclorac dimethylammonium, as component B) benzyl alcohol and glycol and at least two, preferably exactly two, components C different from each other, wherein

the first of the two or more, preferably exactly two, components C is selected from glyphosate including its agriculturally acceptable salts or derivatives; particularly preferred glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium; and the second of the two or more, preferably exactly two, components C is selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium.

In another preferred embodiment of the invention the SL formulation comprises as component A) quinclorac mono-[2(2-hydroxyethoxy)-ethyl]ammonium, as component B) benzyl alcohol and as component C) at least one herbicide selected from the group consisting of
c1) EPSP synthase inhibitors: glyphosate; and
c2) glutamine synthase inhibitors: bilanaphos (bialaphos), glufosinate and glufosinate-P;

including their agriculturally acceptable salts or derivatives;

particularly preferred selected from the group consisting of
c1) EPSP synthase inhibitors: glyphosate, glyphosate-isopropylammonium, glyposate-potassium and glyphosate-trimesium (sulfosate); and
c2) glutamine synthase inhibitors: bilanaphos (bialaphos), bilanaphos-sodium, glufosinate, glufosinate-P, glufosinate-ammonium and glufosinate-P-ammonium;
   especially preferred selected from the group consisting of
c1) EPSP synthase inhibitors: glyphosate, glyphosate-isopropylammonium and glyphosate-trimesium (sulfosate); and
c2) glutamine synthase inhibitors: glufosinate, glufosinate-P, glufosinate-ammonium and glufosinate-P-ammonium;
   more preferred selected from the group consisting of glyphosate-isopropylammonium, glyphosate-trimesium (sulfosate), glufosinate-ammonium and glufosinate-P-ammonium;
   most preferred selected from the group consisting of glyphosate-isopropylammonium and glufosinate-ammonium.

In another preferred embodiment of the invention the SL formulation comprises as component A) quinclorac mono-[2(2-hydroxyethoxy)-ethyl]ammonium, as component B) benzyl alcohol and at least one, preferably exactly one component C selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium.

In another preferred embodiment of the invention the SL formulation comprises as component A) quinclorac mono-[2(2-hydroxyethoxy)-ethyl]ammonium, as component B) benzyl alcohol and at least one, preferably exactly one component C selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium.

In another preferred embodiment of the invention the SL formulation comprises as component A) quinclorac mono-[2(2-hydroxyethoxy)-ethyl]ammonium, as component B) benzyl alcohol and at least two, preferably exactly two, components C different from each other, wherein at least one component C is selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium.

In another preferred embodiment of the invention the SL formulation comprises as component A) quinclorac mono-[2(2-hydroxyethoxy)-ethyl]ammonium, as component B) benzyl alcohol and at least two, preferably exactly two, components C different from each other, wherein at least one component C is selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium.

In another preferred embodiment of the invention the SL formulation comprises as component A) quinclorac mono-[2(2-hydroxyethoxy)-ethyl]ammonium, as component B) benzyl alcohol and at least two, preferably exactly two, components C different from each other, wherein

the first of the two or more, preferably exactly two, components C is selected from glyphosate including its agriculturally acceptable salts or derivatives;
particularly preferred glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium; and
   the second of the two or more, preferably exactly two, components C is selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium.

In another preferred embodiment of the invention the SL formulation comprises as component A) quinclorac mono-[2(2-hydroxyethoxy)-ethyl]ammonium, as component B) glycol and as component C) at least one herbicide selected from the group consisting of c1) EPSP synthase inhibitors: glyphosate; and
c2) glutamine synthase inhibitors: bilanaphos (bialaphos), glufosinate and glufosinate-P;

including their agriculturally acceptable salts or derivatives;

particularly preferred selected from the group consisting of
c1) EPSP synthase inhibitors: glyphosate, glyphosate-isopropylammonium, glyposate-potassium and glyphosate-trimesium (sulfosate); and
c2) glutamine synthase inhibitors: bilanaphos (bialaphos), bilanaphos-sodium, glufosinate, glufosinate-P, glufosinate-ammonium and glufosinate-P-ammonium;
   especially preferred selected from the group consisting of
c1) EPSP synthase inhibitors: glyphosate, glyphosate-isopropylammonium and glyphosate-trimesium (sulfosate); and
c2) glutamine synthase inhibitors: glufosinate, glufosinate-P, glufosinate-ammonium and glufosinate-P-ammonium;
   more preferred selected from the group consisting of glyphosate-isopropylammonium, glyphosate-trimesium (sulfosate), glufosinate-ammonium and glufosinate-P-ammonium;
   most preferred selected from the group consisting of glyphosate-isopropylammonium and glufosinate-ammonium.

In another preferred embodiment of the invention the SL formulation comprises as component A) quinclorac mono-[2(2-hydroxyethoxy)-ethyl]ammonium, as component B) glycol and at least one, preferably exactly one component C selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium.

In another preferred embodiment of the invention the SL formulation comprises as component A) quinclorac mono-[2(2-hydroxyethoxy)-ethyl]ammonium, as component B) glycol and at least one, preferably exactly one component C selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium.

In another preferred embodiment of the invention the SL formulation comprises as component A) quinclorac mono-[2(2-hydroxyethoxy)-ethyl]ammonium, as component B) glycol and at least two, preferably exactly two, components C different from each other, wherein at least one component C is selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium.

In another preferred embodiment of the invention the SL formulation comprises as component A) quinclorac mono-[2(2-hydroxyethoxy)-ethyl]ammonium, as component B) glycol and at least two, preferably exactly two, components C different from each other, wherein at least one component C is selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium.

In another preferred embodiment of the invention the SL formulation comprises as component A) quinclorac mono-[2(2-hydroxyethoxy)-ethyl]ammonium, as component B) glycol and at least two, preferably exactly two, components C different from each other, wherein

the first of the two or more, preferably exactly two, components C is selected from glyphosate including its agriculturally acceptable salts or derivatives;
particularly preferred glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium; and
   the second of the two or more, preferably exactly two, components C is selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium.

In another preferred embodiment of the invention the SL formulation comprises as component A) quinclorac mono-[2(2-hydroxyethoxy)-ethyl]ammonium, as component B) benzyl alcohol and glycol and as component C) at least one herbicide selected from the group consisting of
c1) EPSP synthase inhibitors: glyphosate; and
c2) glutamine synthase inhibitors: bilanaphos (bialaphos), glufosinate and glufosinate-P;

including their agriculturally acceptable salts or derivatives;

particularly preferred selected from the group consisting of
c1) EPSP synthase inhibitors: glyphosate, glyphosate-isopropylammonium, glyposate-potassium and glyphosate-trimesium (sulfosate); and
c2) glutamine synthase inhibitors: bilanaphos (bialaphos), bilanaphos-sodium, glufosinate, glufosinate-P, glufosinate-ammonium and glufosinate-P-ammonium;
   especially preferred selected from the group consisting of
c1) EPSP synthase inhibitors: glyphosate, glyphosate-isopropylammonium and glyphosate-trimesium (sulfosate); and
c2) glutamine synthase inhibitors: glufosinate, glufosinate-P, glufosinate-ammonium and glufosinate-P-ammonium;
   more preferred selected from the group consisting of glyphosate-isopropylammonium, glyphosate-trimesium (sulfosate), glufosinate-ammonium and glufosinate-P-ammonium;
   most preferred selected from the group consisting of glyphosate-isopropylammonium and glufosinate-ammonium.

In another preferred embodiment of the invention the SL formulation comprises as component A) quinclorac mono-[2(2-hydroxyethoxy)-ethyl]ammonium, as component B) benzyl alcohol and glycol and at least one, preferably exactly one component C selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium.

In another preferred embodiment of the invention the SL formulation comprises as component A) quinclorac mono-[2(2-hydroxyethoxy)-ethyl]ammonium, as component B) benzyl alcohol and glycol and at least one, preferably exactly one component C selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium.

In another preferred embodiment of the invention the SL formulation comprises as component A) quinclorac mono-[2(2-hydroxyethoxy)-ethyl]ammonium, as component B) benzyl alcohol and glycol and at least two, preferably exactly two, components C different from each other, wherein at least one component C is selected from the group of
c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium.

In another preferred embodiment of the invention the SL formulation comprises as component A) quinclorac mono-[2(2-hydroxyethoxy)-ethyl]ammonium, as component B) benzyl alcohol and glycol and at least two, preferably exactly two, components C different from each other, wherein at least one component C is selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium.

In another preferred embodiment of the invention the SL formulation comprises as component A) quinclorac mono-[2(2-hydroxyethoxy)-ethyl]ammonium, as component B) benzyl alcohol and glycol and at least two, preferably exactly two, components C different from each other, wherein

the first of the two or more, preferably exactly two, components C is selected from glyphosate including its agriculturally acceptable salts or derivatives;
particularly preferred glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium; and
   the second of the two or more, preferably exactly two, components C is selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium.

The invention the SL formulation may also comprise in addition to components A), B) and C) one or more co-solvents.

Suitable co-solvents are protic solvents, like water, alcohols and polyoles, as well as dipolar aprotic solvents like ethers, ketones, lactones, carbonates, amides and lactames.

Examples for suitable co-solvents are
water;
alcohols like C₁-C₈ alkanoles such as methanol, ethanol, n-propanol, isopropanol, n-butanol, isobutanol, 2-butanol, tert.-butanol, pentanol, isoamyl alcohol, nhexanol, 1-methylpentanol, 1-ethylbutanol, n-octanol, 2-ethylhexanol and the like; C₅-C₈ cycloalkanols such as cyclopentanol, cyclohexanol and the like; polyhydric alcohols, such as sorbitol and the like; alkylene glycol monomethyl ethers such as ethylene glycol monomethyl ether, propylene glycol monomethyl ether and the like; di- and tri-C₂-C₄-alkylene glycol monomethyl ethers such asdiethylene glycol monomethyl ether, dipropylene glycol monomethyl ether and the like;
polyoles like glycerine and the like;
ethers like cyclic ethers which may contain an OH group such as tetrahydrofuran, pyran, dioxan, tetrahydrofurfurol and the like; alkylene glycol dimethyl ethers such as ethylene glycol dimethyl ether, propylene glycol dimethyl ether and the like; di- and tri-C₂-C₄-alkylene glycol dimethyl ethers such as diethylene glycol dimethyl ether, dipropylene glycol dimethyl ether and the like;
ketones, having from 3 to 8 C-atoms and optionally a hydroxyl group, such as acetone, methylethyl ketone, methylpropyl ketone, methyl-4-hydroxybutyl ketone, cyclopentanone, cyclohexanone, diaceton alcohol, mesityloxide;
lactones, having from 3 to 8 C-atoms, such as ⋅-propiolactone, ⋅-butyrolactone,
carbonates, in particular dimethylcarbonat, diethylcarbonat and 2-oxa-1,3-dioxolan;
amides such as dimethylformamide, dimethylacetamid,
lactames, having preferably from 3 to 6 carbon atoms and their N-methyl and Nethyl derivatives, such as pyrrolidin-2-one, N-methylpyrrolidin-2-one, N-ethylpyrrolidin-2-one and the like.

The especially preferred co-solvent is water.

In a preferred embodiment of the invention the SL formulation comprises in addition to components A), B) and C) as defined above, preferably as defined above as preferred, one co-solvent, especially water.

In another preferred embodiment of the invention the SL formulation comprises in addition to components A) and B) as defined above, preferably as defined above as preferred, at least one component C) selected from the group consisting of
c1) EPSP synthase inhibitors: glyphosate; and
c2) glutamine synthase inhibitors: bilanaphos (bialaphos), glufosinate and glufosinate-P;

including their agriculturally acceptable salts or derivatives;

particularly preferred selected from the group consisting of
c1) EPSP synthase inhibitors: glyphosate, glyphosate-isopropylammonium, glyposate-potassium and glyphosate-trimesium (sulfosate); and
c2) glutamine synthase inhibitors: bilanaphos (bialaphos), bilanaphos-sodium, glufosinate, glufosinate-P, glufosinate-ammonium and glufosinate-P-ammonium;
   especially preferred selected from the group consisting of
c1) EPSP synthase inhibitors: glyphosate, glyphosate-isopropylammonium and glyphosate-trimesium (sulfosate); and
c2) glutamine synthase inhibitors: glufosinate, glufosinate-P, glufosinate-ammonium and glufosinate-P-ammonium;
   more preferred selected from the group consisting of glyphosate-isopropylammonium, glyphosate-trimesium (sulfosate), glufosinate-ammonium and glufosinate-P-ammonium;
   most preferred selected from the group consisting of glyphosate-isopropylammonium and glufosinate-ammonium;
   and one co-solvent, especially water.

In another preferred embodiment of the invention the SL formulation comprises as component A) quinclorac dimethylammonium, as component B) benzyl alcohol, and as component C) at least one herbicide selected from the group consisting of
c1) EPSP synthase inhibitors: glyphosate; and
c2) glutamine synthase inhibitors: bilanaphos (bialaphos), glufosinate and glufosinate-P;

including their agriculturally acceptable salts or derivatives;

particularly preferred selected from the group consisting of
c1) EPSP synthase inhibitors: glyphosate, glyphosate-isopropylammonium, glyposate-potassium and glyphosate-trimesium (sulfosate); and
c2) glutamine synthase inhibitors: bilanaphos (bialaphos), bilanaphos-sodium, glufosinate, glufosinate-P, glufosinate-ammonium and glufosinate-P-ammonium;
   especially preferred selected from the group consisting of
c1) EPSP synthase inhibitors: glyphosate, glyphosate-isopropylammonium and glyphosate-trimesium (sulfosate); and
c2) glutamine synthase inhibitors: glufosinate, glufosinate-P, glufosinate-ammonium and glufosinate-P-ammonium;
   more preferred selected from the group consisting of glyphosate-isopropylammonium, glyphosate-trimesium (sulfosate), glufosinate-ammonium and glufosinate-P-ammonium;
   most preferred selected from the group consisting of glyphosate-isopropylammonium and glufosinate-ammonium;
   and one co-solvent, especially water.

In another preferred embodiment of the invention the SL formulation comprises as component A) quinclorac dimethylammonium, as component B) benzyl alcohol, and at least one, preferably exactly one component C selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium;
   and one co-solvent, especially water.

In another preferred embodiment of the invention the SL formulation comprises as component A) quinclorac dimethylammonium, as component B) benzyl alcohol, and at least one, preferably exactly one component C selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   and one co-solvent, especially water.

In another preferred embodiment of the invention the SL formulation comprises as component A) quinclorac dimethylammonium, as component B) benzyl alcohol, and at least two, preferably exactly two, components C different from each other, wherein at least one component C is selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium;
   and one co-solvent, especially water.

In another preferred embodiment of the invention the SL formulation comprises as component A) quinclorac dimethylammonium, as component B) benzyl alcohol, and at least two, preferably exactly two, components C different from each other, wherein at least one component C is selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   and one co-solvent, especially water.

In another preferred embodiment of the invention the SL formulation comprises as component A) quinclorac dimethylammonium, as component B) benzyl alcohol, and at least two, preferably exactly two, components C different from each other, wherein the first of the two or more, preferably exactly two, components C is selected from glyphosate including its agriculturally acceptable salts or derivatives;
particularly preferred glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium; and
   and the second of the two or more, preferably exactly two, components C is selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   and one co-solvent, especially water.

In another preferred embodiment of the invention the SL formulation comprises as component A) quinclorac dimethylammonium, as component B) glycol, and as component C) at least one herbicide selected from the group consisting of
c1) EPSP synthase inhibitors: glyphosate; and
c2) glutamine synthase inhibitors: bilanaphos (bialaphos), glufosinate and glufosinate-P;

including their agriculturally acceptable salts or derivatives;
particularly preferred selected from the group consisting of
c1) EPSP synthase inhibitors: glyphosate, glyphosate-isopropylammonium, glyposate-potassium and glyphosate-trimesium (sulfosate); and
c2) glutamine synthase inhibitors: bilanaphos (bialaphos), bilanaphos-sodium, glufosinate, glufosinate-P, glufosinate-ammonium and glufosinate-P-ammonium;
   especially preferred selected from the group consisting of
c1) EPSP synthase inhibitors: glyphosate, glyphosate-isopropylammonium and glyphosate-trimesium (sulfosate); and
c2) glutamine synthase inhibitors: glufosinate, glufosinate-P, glufosinate-ammonium and glufosinate-P-ammonium;
   more preferred selected from the group consisting of glyphosate-isopropylammonium, glyphosate-trimesium (sulfosate), glufosinate-ammonium and glufosinate-P-ammonium;
   most preferred selected from the group consisting of glyphosate-isopropylammonium and glufosinate-ammonium;
   and one co-solvent, especially water.

In another preferred embodiment of the invention the SL formulation comprises as component A) quinclorac dimethylammonium, as component B) glycol, and at least one, preferably exactly one component C selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium;
   and one co-solvent, especially water.

In another preferred embodiment of the invention the SL formulation comprises as component A) quinclorac dimethylammonium, as component B) glycol, and at least one, preferably exactly one component C selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   and one co-solvent, especially water.

In another preferred embodiment of the invention the SL formulation comprises as component A) quinclorac dimethylammonium, as component B) glycol, and at least two, preferably exactly two, components C different from each other, wherein at least one component C is selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium;
   and one co-solvent, especially water.

In another preferred embodiment of the invention the SL formulation comprises as component A) quinclorac dimethylammonium, as component B) glycol, and at least two, preferably exactly two, components C different from each other, wherein at least one component C is selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   and one co-solvent, especially water.

In another preferred embodiment of the invention the SL formulation comprises as component A) quinclorac dimethylammonium, as component B) glycol, and at least two, preferably exactly two, components C different from each other, wherein the first of the two or more, preferably exactly two, components C is selected from glyphosate including its agriculturally acceptable salts or derivatives;
particularly preferred glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium; and
   the second of the two or more, preferably exactly two, components C is selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   and one co-solvent, especially water.

In another preferred embodiment of the invention the SL formulation comprises as component A) quinclorac dimethylammonium, as component B) benzyl alcohol and glycol, and as component C) at least one herbicide selected from the group consisting of
c1) EPSP synthase inhibitors: glyphosate; and
c2) glutamine synthase inhibitors: bilanaphos (bialaphos), glufosinate and glufosinate-P;

including their agriculturally acceptable salts or derivatives;

particularly preferred selected from the group consisting of
c1) EPSP synthase inhibitors: glyphosate, glyphosate-isopropylammonium, glyposate-potassium and glyphosate-trimesium (sulfosate); and
c2) glutamine synthase inhibitors: bilanaphos (bialaphos), bilanaphos-sodium, glufosinate, glufosinate-P, glufosinate-ammonium and glufosinate-P-ammonium;
   especially preferred selected from the group consisting of
c1) EPSP synthase inhibitors: glyphosate, glyphosate-isopropylammonium and glyphosate-trimesium (sulfosate); and
c2) glutamine synthase inhibitors: glufosinate, glufosinate-P, glufosinate-ammonium and glufosinate-P-ammonium;
   more preferred selected from the group consisting of glyphosate-isopropylammonium, glyphosate-trimesium (sulfosate), glufosinate-ammonium and glufosinate-P-ammonium;
   most preferred selected from the group consisting of glyphosate-isopropylammonium and glufosinate-ammonium;
   and one co-solvent, especially water.

In another preferred embodiment of the invention the SL formulation comprises as component A) quinclorac dimethylammonium, as component B) benzyl alcohol and glycol, and at least one, preferably exactly one component C selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium;
   and one co-solvent, especially water.

In another preferred embodiment of the invention the SL formulation comprises as component A) quinclorac dimethylammonium, as component B) benzyl alcohol and glycol, and at least one, preferably exactly one component C selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   and one co-solvent, especially water.

In another preferred embodiment of the invention the SL formulation comprises as component A) quinclorac dimethylammonium, as component B) benzyl alcohol and glycol, and at least two, preferably exactly two, components C different from each other, wherein at least one component C is selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium;
   and one co-solvent, especially water.

In another preferred embodiment of the invention the SL formulation comprises as component A) quinclorac dimethylammonium, as component B) benzyl alcohol and glycol, and at least two, preferably exactly two, components C different from each other, wherein at least one component C is selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   and one co-solvent, especially water.

In another preferred embodiment of the invention the SL formulation comprises as component A) quinclorac dimethylammonium, as component B) benzyl alcohol and glycol, and at least two, preferably exactly two, components C different from each other, wherein

the first of the two or more, preferably exactly two, components C is selected from glyphosate including its agriculturally acceptable salts or derivatives;
particularly preferred glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium; and
   the second of the two or more, preferably exactly two, components C is selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   and one co-solvent, especially water.

In another preferred embodiment of the invention the SL formulation comprises as component A) quinclorac mono-[2(2-hydroxyethoxy)-ethyl]ammonium, as component B) benzyl alcohol, and as component C) at least one herbicide selected from the group consisting of
c1) EPSP synthase inhibitors: glyphosate; and
c2) glutamine synthase inhibitors: bilanaphos (bialaphos), glufosinate and glufosinate-P;

including their agriculturally acceptable salts or derivatives;

particularly preferred selected from the group consisting of
c1) EPSP synthase inhibitors: glyphosate, glyphosate-isopropylammonium, glyposate-potassium and glyphosate-trimesium (sulfosate); and
c2) glutamine synthase inhibitors: bilanaphos (bialaphos), bilanaphos-sodium, glufosinate, glufosinate-P, glufosinate-ammonium and glufosinate-P-ammonium;
   especially preferred selected from the group consisting of
c1) EPSP synthase inhibitors: glyphosate, glyphosate-isopropylammonium and glyphosate-trimesium (sulfosate); and
c2) glutamine synthase inhibitors: glufosinate, glufosinate-P, glufosinate-ammonium and glufosinate-P-ammonium;
   more preferred selected from the group consisting of glyphosate-isopropylammonium, glyphosate-trimesium (sulfosate), glufosinate-ammonium and glufosinate-P-ammonium;
   most preferred selected from the group consisting of glyphosate-isopropylammonium and glufosinate-ammonium;
   and one co-solvent, especially water.

In another preferred embodiment of the invention the SL formulation comprises as component A) quinclorac mono-[2(2-hydroxyethoxy)-ethyl]ammonium, as component B) benzyl alcohol, and at least one, preferably exactly one component C selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium;
   and one co-solvent, especially water.

In another preferred embodiment of the invention the SL formulation comprises as component A) quinclorac mono-[2(2-hydroxyethoxy)-ethyl]ammonium, as component B) benzyl alcohol, and at least one, preferably exactly one component C selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   and one co-solvent, especially water.

In another preferred embodiment of the invention the SL formulation comprises as component A) quinclorac mono-[2(2-hydroxyethoxy)-ethyl]ammonium, as component B) benzyl alcohol, and at least two, preferably exactly two, components C different from each other, wherein at least one component C is selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium;
   and one co-solvent, especially water.

In another preferred embodiment of the invention the SL formulation comprises as component A) quinclorac mono-[2(2-hydroxyethoxy)-ethyl]ammonium, as component B) benzyl alcohol, and at least two, preferably exactly two, components C different from each other, wherein at least one component C is selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   and one co-solvent, especially water.

In another preferred embodiment of the invention the SL formulation comprises as component A) quinclorac mono-[2(2-hydroxyethoxy)-ethyl]ammonium, as component B) benzyl alcohol, and at least two, preferably exactly two, components C different from each other, wherein

the first of the two or more, preferably exactly two, components C is selected from glyphosate including its agriculturally acceptable salts or derivatives;
particularly preferred glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium; and
   the second of the two or more, preferably exactly two, components C is selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   and one co-solvent, especially water.

In another preferred embodiment of the invention the SL formulation comprises as component A) quinclorac mono-[2(2-hydroxyethoxy)-ethyl]ammonium, as component B) glycol, and as component C) at least one herbicide selected from the group consisting of c1) EPSP synthase inhibitors: glyphosate; and
c2) glutamine synthase inhibitors: bilanaphos (bialaphos), glufosinate and glufosinate-P;

including their agriculturally acceptable salts or derivatives;

particularly preferred selected from the group consisting of
c1) EPSP synthase inhibitors: glyphosate, glyphosate-isopropylammonium, glyposate-potassium and glyphosate-trimesium (sulfosate); and
c2) glutamine synthase inhibitors: bilanaphos (bialaphos), bilanaphos-sodium, glufosinate, glufosinate-P, glufosinate-ammonium and glufosinate-P-ammonium;
   especially preferred selected from the group consisting of
c1) EPSP synthase inhibitors: glyphosate, glyphosate-isopropylammonium and glyphosate-trimesium (sulfosate); and
c2) glutamine synthase inhibitors: glufosinate, glufosinate-P, glufosinate-ammonium and glufosinate-P-ammonium;
   more preferred selected from the group consisting of glyphosate-isopropylammonium, glyphosate-trimesium (sulfosate), glufosinate-ammonium and glufosinate-P-ammonium;
   most preferred selected from the group consisting of glyphosate-isopropylammonium and glufosinate-ammonium;
   and one co-solvent, especially water.

In another preferred embodiment of the invention the SL formulation comprises as component A) quinclorac mono-[2(2-hydroxyethoxy)-ethyl]ammonium, as component B) glycol, and at least one, preferably exactly one component C selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium;
   and one co-solvent, especially water.

In another preferred embodiment of the invention the SL formulation comprises as component A) quinclorac mono-[2(2-hydroxyethoxy)-ethyl]ammonium, as component B) glycol, and at least one, preferably exactly one component C selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   and one co-solvent, especially water.

In another preferred embodiment of the invention the SL formulation comprises as component A) quinclorac mono-[2(2-hydroxyethoxy)-ethyl]ammonium, as component B) glycol, and at least two, preferably exactly two, components C different from each other, wherein at least one component C is selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium;
   and one co-solvent, especially water.

In another preferred embodiment of the invention the SL formulation comprises as component A) quinclorac mono-[2(2-hydroxyethoxy)-ethyl]ammonium, as component B) glycol, and at least two, preferably exactly two, components C different from each other, wherein at least one component C is selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   and one co-solvent, especially water.

In another preferred embodiment of the invention the SL formulation comprises as component A) quinclorac mono-[2(2-hydroxyethoxy)-ethyl]ammonium, as component B) glycol, and at least two, preferably exactly two, components C different from each other, wherein

the first of the two or more, preferably exactly two, components C is selected from glyphosate including its agriculturally acceptable salts or derivatives;
particularly preferred glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium; and
   the second of the two or more, preferably exactly two, components C is selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   and one co-solvent, especially water.

In another preferred embodiment of the invention the SL formulation comprises as component A) quinclorac mono-[2(2-hydroxyethoxy)-ethyl]ammonium, as component B) benzyl alcohol and glycol, and as component C) at least one herbicide selected from the group consisting of
c1) EPSP synthase inhibitors: glyphosate; and
c2) glutamine synthase inhibitors: bilanaphos (bialaphos), glufosinate and glufosinate-P;

including their agriculturally acceptable salts or derivatives;
particularly preferred selected from the group consisting of
c1) EPSP synthase inhibitors: glyphosate, glyphosate-isopropylammonium, glyposate-potassium and glyphosate-trimesium (sulfosate); and
c2) glutamine synthase inhibitors: bilanaphos (bialaphos), bilanaphos-sodium, glufosinate, glufosinate-P, glufosinate-ammonium and glufosinate-P-ammonium;
   especially preferred selected from the group consisting of
c1) EPSP synthase inhibitors: glyphosate, glyphosate-isopropylammonium and glyphosate-trimesium (sulfosate); and
c2) glutamine synthase inhibitors: glufosinate, glufosinate-P, glufosinate-ammonium and glufosinate-P-ammonium;
   more preferred selected from the group consisting of glyphosate-isopropylammonium, glyphosate-trimesium (sulfosate), glufosinate-ammonium and glufosinate-P-ammonium;
   most preferred selected from the group consisting of glyphosate-isopropylammonium and glufosinate-ammonium;
   and one co-solvent, especially water.

In another preferred embodiment of the invention the SL formulation comprises as component A) quinclorac mono-[2(2-hydroxyethoxy)-ethyl]ammonium, as component B) benzyl alcohol and glycol, and at least one, preferably exactly one component C selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium;
   and one co-solvent, especially water.

In another preferred embodiment of the invention the SL formulation comprises as component A) quinclorac mono-[2(2-hydroxyethoxy)-ethyl]ammonium, as component B) benzyl alcohol and glycol, and at least one, preferably exactly one component C selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   and one co-solvent, especially water.

In another preferred embodiment of the invention the SL formulation comprises as component A) quinclorac mono-[2(2-hydroxyethoxy)-ethyl]ammonium, as component B) benzyl alcohol and glycol, and at least two, preferably exactly two, components C different from each other, wherein at least one component C is selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium;
   and one co-solvent, especially water.

In another preferred embodiment of the invention the SL formulation comprises as component A) quinclorac mono-[2(2-hydroxyethoxy)-ethyl]ammonium, as component B) benzyl alcohol and glycol, and at least two, preferably exactly two, components C different from each other, wherein at least one component C is selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   and one co-solvent, especially water.

In another preferred embodiment of the invention the SL formulation comprises as component A) quinclorac mono-[2(2-hydroxyethoxy)-ethyl]ammonium, as component B) benzyl alcohol and glycol, and at least two, preferably exactly two, components C different from each other, wherein

the first of the two or more, preferably exactly two, components C is selected from glyphosate including its agriculturally acceptable salts or derivatives;
particularly preferred glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium; and
   the second of the two or more, preferably exactly two, components C is selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   and one co-solvent, especially water.

The SL formulation of the invention may also comprise in addition to components A), B) and C) one or more formulation additives, such as surfactants, defoamers, preservatives, colorants, stabilizers and the like.

Suitable surfactants are

anionic surfactants such as the alkali metal salts, alkaline earth metal salts, in particular the sodium, potassium and calcium salts, and ammonium salts of
alkylsulfonates, such as lauryl sulfonate, isotridecylsulfonate,
alkylsulfates, in particular fatty alcohol sulfates, such as lauryl sulfate, isotridecylsulfate, cetylsulfate, stearylsulfate;
aryl- and alkylarylsulfonates, such as napthylsulfonate, dibutylnaphtylsulfonate, dodecyldiphenylether sulfonate, cumylsulfonate, nonylbenzenesulfonate, dodecylbenzene sulfonate;
sulfonates of fatty acids and fatty acid esters;
sulfates of fatty acids and fatty acid esters;
sulfates of ethoxylated alkanols, such as sulfates of ethoxylated lauryl alcohol;
sulfates of alkoxylated alkylphenols;
alkylphosphates, C₈-C₁₆ alkylphosphates;
dialkylphosphates, C₈-C₁₆ dialkylphosphates;
dialkylesters of sulfosuccinic acid, such as dioctylsulfosuccinate,
acylsarcosinates,
fatty acids, such as stearates,
acylglutamates,
ligninsulfonates,
condensates of naphthalinesulfonic acid or phenolsulfonic acid with formaldehyde;
phosphate esters of alkoxylated alcanols or block copolymers based on EO/PO;
   non-ionic surfactants, such as
ethoxylated alkanols, in particular ethoxylated fatty alcohols and ethoxylated oxoalcohols, such as ethoxylated lauryl alcohol, ethoxylated isotridecanol, ethoxylated cetyl alcohol, ethoxylated stearyl alcohol, and esters thereof, such as acetates;
ethoxylated alkylphenols, such as ethoxylated nonylphenyl, ethoxylated dodecylphenyl, ethoxylated isotridecylphenol and the esters thereof, e.g. the acetates;
alkylglucosides and alkyl polygucosides;
ethoxylated alkylglucosides;
ethoxylated fatty amines;
ethoxylated fatty acids;
partial esters, such as mono-, di- and triesters of fatty acids with glycerin or Sorbitan, such as glycerin monostearate, sorbitanmonooleat, sorbitantristearate;
ethoxylated esters of fatty acids with glycerin or sorbitan, such as ethoxylated glycerin monostearate;
ethoxylates of vegetable oils or animal fats, such as corn oil ethoxylate, castor oil ethoxylate, tallow oil ethoxylate;
ethoxylates of fatty amines, fatty amides or of fatty acid diethanolamides;
alkoxylated EO/PO polymers;
   and
   cationic surfactants, such as
quaterary ammonium compounds, in particular alkyltrimethylammonium salts and dialkyldimethylammonium salts, e.g. the halides, sulfates and alkylsulfates;
pyridinium salts, in particular alkylpyridinium salts e.g. the halides, sulfates and C₁-C₄-alkylsulfates; and
imidazolinium salts in particular N,N′-dialkylimidazolinium salts, e.g. the halides, sulfates or methoxulfates;
   or mixtures thereof.

Suitable defoamers include polysiloxanes, such as polydimethyl siloxane.

Suitable preservatives to prevent microbial spoiling of the compositions of the invention include formaldehyde, alkyl esters of p-hydroxybenzoic acid, sodium benzoate, 2-bromo-2-nitropropane-1,3-diol, o-phenylphenol, thiazolinones, such as benzisothiazolinone, 5-chloro-2-methyl-4-isothiazolinone, pentachlorophenol, 2,4-dichlorobenzyl alcohol and mixtures thereof.

Suitable stabilizers comprise e.g. UV-absorbers such as cinnamic esters, 3,3-diphenyl-2-cyano acrylates, hydroxy and/or alkoxy substituted benzophenones, N-(hydroxylphenyl)-benzotriazoles, hydroxyphenyl-s-triazines, oxalic amides and salicylates, e.g. the UVINUL® 3000, 3008, 3040, 3048, 3049, 3050, 3030, 3035, 3039, 3088, UVINUL® MC80 and radical scavengers, e.g. ascorbic acid, sterically hindered amines (HALS-compounds) such as UVINUL® 4049H, 4050H and 5050H, and the like and anti-oxidants such as vitamin E.

In a preferred embodiment of the invention the SL formulation comprises in addition to components A), B) and C) as defined above, preferably as defined above as preferred, one or more formulation additives, especially one surfactant.

In another preferred embodiment of the invention the SL formulation comprises in addition to components A) and B) as defined above, preferably as defined above as preferred, at least one component C) selected from the group consisting of
c1) EPSP synthase inhibitors: glyphosate; and
c2) glutamine synthase inhibitors: bilanaphos (bialaphos), glufosinate and glufosinate-P;

including their agriculturally acceptable salts or derivatives;
particularly preferred selected from the group consisting of
c1) EPSP synthase inhibitors: glyphosate, glyphosate-isopropylammonium, glyposate-potassium and glyphosate-trimesium (sulfosate); and
c2) glutamine synthase inhibitors: bilanaphos (bialaphos), bilanaphos-sodium, glufosinate, glufosinate-P, glufosinate-ammonium and glufosinate-P-ammonium;
   especially preferred selected from the group consisting of
c1) EPSP synthase inhibitors: glyphosate, glyphosate-isopropylammonium and glyphosate-trimesium (sulfosate); and
c2) glutamine synthase inhibitors: glufosinate, glufosinate-P, glufosinate-ammonium and glufosinate-P-ammonium;
   more preferred selected from the group consisting of glyphosate-isopropylammonium, glyphosate-trimesium (sulfosate), glufosinate-ammonium and glufosinate-P-ammonium;
   most preferred selected from the group consisting of glyphosate-isopropylammonium and glufosinate-ammonium;
   and one or more formulation additives, especially one surfactant.

In another preferred embodiment of the invention the SL formulation comprises as component A) quinclorac dimethylammonium, as component B) benzyl alcohol, and as component C) at least one herbicide selected from the group consisting of
c1) EPSP synthase inhibitors: glyphosate; and
c2) glutamine synthase inhibitors: bilanaphos (bialaphos), glufosinate and glufosinate-P;

including their agriculturally acceptable salts or derivatives;

particularly preferred selected from the group consisting of
c1) EPSP synthase inhibitors: glyphosate, glyphosate-isopropylammonium, glyposate-potassium and glyphosate-trimesium (sulfosate); and
c2) glutamine synthase inhibitors: bilanaphos (bialaphos), bilanaphos-sodium, glufosinate, glufosinate-P, glufosinate-ammonium and glufosinate-P-ammonium;
   especially preferred selected from the group consisting of
c1) EPSP synthase inhibitors: glyphosate, glyphosate-isopropylammonium and glyphosate-trimesium (sulfosate); and
c2) glutamine synthase inhibitors: glufosinate, glufosinate-P, glufosinate-ammonium and glufosinate-P-ammonium;
   more preferred selected from the group consisting of glyphosate-isopropylammonium, glyphosate-trimesium (sulfosate), glufosinate-ammonium and glufosinate-P-ammonium;
   most preferred selected from the group consisting of glyphosate-isopropylammonium and glufosinate-ammonium;
   and one or more formulation additives, especially one surfactant.

In another preferred embodiment of the invention the SL formulation comprises as component A) quinclorac dimethylammonium, as component B) benzyl alcohol, and at least one, preferably exactly one component C selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium;
   and one or more formulation additives, especially one surfactant.

In another preferred embodiment of the invention the SL formulation comprises as component A) quinclorac dimethylammonium, as component B) benzyl alcohol, and at least one, preferably exactly one component C selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   and one or more formulation additives, especially one surfactant.

In another preferred embodiment of the invention the SL formulation comprises as component A) quinclorac dimethylammonium, as component B) benzyl alcohol, and at least two, preferably exactly two, components C different from each other, wherein at least one component C is selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   and one or more formulation additives, especially one surfactant.

In another preferred embodiment of the invention the SL formulation comprises as component A) quinclorac dimethylammonium, as component B) benzyl alcohol, and at least two, preferably exactly two, components C different from each other, wherein the first of the two or more, preferably exactly two, components C is selected from glyphosate including its agriculturally acceptable salts or derivatives;
particularly preferred glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium; and
   the second of the two or more, preferably exactly two, components C is selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   and one or more formulation additives, especially one surfactant.

In another preferred embodiment of the invention the SL formulation comprises as component A) quinclorac dimethylammonium, as component B) glycol, and as component C) at least one herbicide selected from the group consisting of
c1) EPSP synthase inhibitors: glyphosate; and
c2) glutamine synthase inhibitors: bilanaphos (bialaphos), glufosinate and glufosinate-P;

including their agriculturally acceptable salts or derivatives;

particularly preferred selected from the group consisting of
c1) EPSP synthase inhibitors: glyphosate, glyphosate-isopropylammonium, glyposate-potassium and glyphosate-trimesium (sulfosate); and
c2) glutamine synthase inhibitors: bilanaphos (bialaphos), bilanaphos-sodium, glufosinate, glufosinate-P, glufosinate-ammonium and glufosinate-P-ammonium;
   especially preferred selected from the group consisting of
c1) EPSP synthase inhibitors: glyphosate, glyphosate-isopropylammonium and glyphosate-trimesium (sulfosate); and
c2) glutamine synthase inhibitors: glufosinate, glufosinate-P, glufosinate-ammonium and glufosinate-P-ammonium;
   more preferred selected from the group consisting of glyphosate-isopropylammonium, glyphosate-trimesium (sulfosate), glufosinate-ammonium and glufosinate-P-ammonium;
   most preferred selected from the group consisting of glyphosate-isopropylammonium and glufosinate-ammonium;
   and one or more formulation additives, especially one surfactant.

In another preferred embodiment of the invention the SL formulation comprises as component A) quinclorac dimethylammonium, as component B) glycol, and at least one, preferably exactly one component C selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium;
   and one or more formulation additives, especially one surfactant.

In another preferred embodiment of the invention the SL formulation comprises as component A) quinclorac dimethylammonium, as component B) glycol, and at least one, preferably exactly one component C selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   and one or more formulation additives, especially one surfactant.

In another preferred embodiment of the invention the SL formulation comprises as component A) quinclorac dimethylammonium, as component B) glycol, and at least two, preferably exactly two, components C different from each other, wherein at least one component C is selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium;
   and one or more formulation additives, especially one surfactant.

In another preferred embodiment of the invention the SL formulation comprises as component A) quinclorac dimethylammonium, as component B) glycol, and at least two, preferably exactly two, components C different from each other, wherein at least one component C is selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   and one or more formulation additives, especially one surfactant.

In another preferred embodiment of the invention the SL formulation comprises as component A) quinclorac dimethylammonium, as component B) glycol, and at least two, preferably exactly two, components C different from each other, wherein the first of the two or more, preferably exactly two, components C is selected from glyphosate including its agriculturally acceptable salts or derivatives;
particularly preferred glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium; and
   the second of the two or more, preferably exactly two, components C is selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   and one or more formulation additives, especially one surfactant.

In another preferred embodiment of the invention the SL formulation comprises as component A) quinclorac dimethylammonium, as component B) benzyl alcohol and glycol, and as component C) at least one herbicide selected from the group consisting of
c1) EPSP synthase inhibitors: glyphosate; and
c2) glutamine synthase inhibitors: bilanaphos (bialaphos), glufosinate and glufosinate-P;

including their agriculturally acceptable salts or derivatives;

particularly preferred selected from the group consisting of
c1) EPSP synthase inhibitors: glyphosate, glyphosate-isopropylammonium, glyposate-potassium and glyphosate-trimesium (sulfosate); and
c2) glutamine synthase inhibitors: bilanaphos (bialaphos), bilanaphos-sodium, glufosinate, glufosinate-P, glufosinate-ammonium and glufosinate-P-ammonium;
   especially preferred selected from the group consisting of
c1) EPSP synthase inhibitors: glyphosate, glyphosate-isopropylammonium and glyphosate-trimesium (sulfosate); and
c2) glutamine synthase inhibitors: glufosinate, glufosinate-P, glufosinate-ammonium and glufosinate-P-ammonium;
   more preferred selected from the group consisting of glyphosate-isopropylammonium, glyphosate-trimesium (sulfosate), glufosinate-ammonium and glufosinate-P-ammonium;
   most preferred selected from the group consisting of glyphosate-isopropylammonium and glufosinate-ammonium;
   and one or more formulation additives, especially one surfactant.

In another preferred embodiment of the invention the SL formulation comprises as component A) quinclorac dimethylammonium, as component B) benzyl alcohol and glycol, and at least one, preferably exactly one component C selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium;
   and one or more formulation additives, especially one surfactant.

In another preferred embodiment of the invention the SL formulation comprises as component A) quinclorac dimethylammonium, as component B) benzyl alcohol and glycol, and at least one, preferably exactly one component C selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   and one or more formulation additives, especially one surfactant.

In another preferred embodiment of the invention the SL formulation comprises as component A) quinclorac dimethylammonium, as component B) benzyl alcohol and glycol, and at least two, preferably exactly two, components C different from each other, wherein at least one component C is selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium;
   and one or more formulation additives, especially one surfactant.

In another preferred embodiment of the invention the SL formulation comprises as component A) quinclorac dimethylammonium, as component B) benzyl alcohol and glycol, and at least two, preferably exactly two, components C different from each other, wherein at least one component C is selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   and one or more formulation additives, especially one surfactant.

In another preferred embodiment of the invention the SL formulation comprises as component A) quinclorac dimethylammonium, as component B) benzyl alcohol and glycol, and at least two, preferably exactly two, components C different from each other, wherein

the first of the two or more, preferably exactly two, components C is selected from glyphosate including its agriculturally acceptable salts or derivatives;
particularly preferred glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium; and
   the second of the two or more, preferably exactly two, components C is selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   and one or more formulation additives, especially one surfactant.

In another preferred embodiment of the invention the SL formulation comprises as component A) quinclorac mono-[2(2-hydroxyethoxy)-ethyl]ammonium, as component B) benzyl alcohol, and as component C) at least one herbicide selected from the group consisting of
c1) EPSP synthase inhibitors: glyphosate; and
c2) glutamine synthase inhibitors: bilanaphos (bialaphos), glufosinate and glufosinate-P;

including their agriculturally acceptable salts or derivatives;

particularly preferred selected from the group consisting of
c1) EPSP synthase inhibitors: glyphosate, glyphosate-isopropylammonium, glyposate-potassium and glyphosate-trimesium (sulfosate); and
c2) glutamine synthase inhibitors: bilanaphos (bialaphos), bilanaphos-sodium, glufosinate, glufosinate-P, glufosinate-ammonium and glufosinate-P-ammonium;
   especially preferred selected from the group consisting of
c1) EPSP synthase inhibitors: glyphosate, glyphosate-isopropylammonium and glyphosate-trimesium (sulfosate); and
c2) glutamine synthase inhibitors: glufosinate, glufosinate-P, glufosinate-ammonium and glufosinate-P-ammonium;
   more preferred selected from the group consisting of glyphosate-isopropylammonium, glyphosate-trimesium (sulfosate), glufosinate-ammonium and glufosinate-P-ammonium;
   most preferred selected from the group consisting of glyphosate-isopropylammonium and glufosinate-ammonium;
   and one or more formulation additives, especially one surfactant.

In another preferred embodiment of the invention the SL formulation comprises as component A) quinclorac mono-[2(2-hydroxyethoxy)-ethyl]ammonium, as component B) benzyl alcohol, and at least one, preferably exactly one component C selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium;
   and one or more formulation additives, especially one surfactant.

In another preferred embodiment of the invention the SL formulation comprises as component A) quinclorac mono-[2(2-hydroxyethoxy)-ethyl]ammonium, as component B) benzyl alcohol, and at least one, preferably exactly one component C selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   and one or more formulation additives, especially one surfactant.

In another preferred embodiment of the invention the SL formulation comprises as component A) quinclorac mono-[2(2-hydroxyethoxy)-ethyl]ammonium, as component B) benzyl alcohol, and at least two, preferably exactly two, components C different from each other, wherein at least one component C is selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium;
   and one or more formulation additives, especially one surfactant.

In another preferred embodiment of the invention the SL formulation comprises as component A) quinclorac mono-[2(2-hydroxyethoxy)-ethyl]ammonium, as component B) benzyl alcohol, and at least two, preferably exactly two, components C different from each other, wherein at least one component C is selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   and one or more formulation additives, especially one surfactant.

In another preferred embodiment of the invention the SL formulation comprises as component A) quinclorac mono-[2(2-hydroxyethoxy)-ethyl]ammonium, as component B) benzyl alcohol, and at least two, preferably exactly two, components C different from each other, wherein

the first of the two or more, preferably exactly two, components C is selected from glyphosate including its agriculturally acceptable salts or derivatives;
particularly preferred glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium; and
   the second of the two or more, preferably exactly two, components C is selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   and one or more formulation additives, especially one surfactant.

In another preferred embodiment of the invention the SL formulation comprises as component A) quinclorac mono-[2(2-hydroxyethoxy)-ethyl]ammonium, as component B) glycol, and as component C) at least one herbicide selected from the group consisting of c1) EPSP synthase inhibitors: glyphosate; and
c2) glutamine synthase inhibitors: bilanaphos (bialaphos), glufosinate and glufosinate-P;

including their agriculturally acceptable salts or derivatives;

particularly preferred selected from the group consisting of
c1) EPSP synthase inhibitors: glyphosate, glyphosate-isopropylammonium, glyposate-potassium and glyphosate-trimesium (sulfosate); and
c2) glutamine synthase inhibitors: bilanaphos (bialaphos), bilanaphos-sodium, glufosinate, glufosinate-P, glufosinate-ammonium and glufosinate-P-ammonium;
   especially preferred selected from the group consisting of
c1) EPSP synthase inhibitors: glyphosate, glyphosate-isopropylammonium and glyphosate-trimesium (sulfosate); and
c2) glutamine synthase inhibitors: glufosinate, glufosinate-P, glufosinate-ammonium and glufosinate-P-ammonium;
   more preferred selected from the group consisting of glyphosate-isopropylammonium, glyphosate-trimesium (sulfosate), glufosinate-ammonium and glufosinate-P-ammonium;
   most preferred selected from the group consisting of glyphosate-isopropylammonium and glufosinate-ammonium;
   and one or more formulation additives, especially one surfactant.

In another preferred embodiment of the invention the SL formulation comprises as component A) quinclorac mono-[2(2-hydroxyethoxy)-ethyl]ammonium, as component B) glycol, and at least one, preferably exactly one component C selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium;
   and one or more formulation additives, especially one surfactant.

In another preferred embodiment of the invention the SL formulation comprises as component A) quinclorac mono-[2(2-hydroxyethoxy)-ethyl]ammonium, as component B) glycol, and at least one, preferably exactly one component C selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   and one or more formulation additives, especially one surfactant.

In another preferred embodiment of the invention the SL formulation comprises as component A) quinclorac mono-[2(2-hydroxyethoxy)-ethyl]ammonium, as component B) glycol, and at least two, preferably exactly two, components C different from each other, wherein at least one component C is selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium;
   and one or more formulation additives, especially one surfactant.

In another preferred embodiment of the invention the SL formulation comprises as component A) quinclorac mono-[2(2-hydroxyethoxy)-ethyl]ammonium, as component B) glycol, and at least two, preferably exactly two, components C different from each other, wherein at least one component C is selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   and one or more formulation additives, especially one surfactant.

In another preferred embodiment of the invention the SL formulation comprises as component A) quinclorac mono-[2(2-hydroxyethoxy)-ethyl]ammonium, as component B) glycol, and at least two, preferably exactly two, components C different from each other, wherein the first of the two or more, preferably exactly two, components C is selected from glyphosate including its agriculturally acceptable salts or derivatives;
particularly preferred glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium; and
   the second of the two or more, preferably exactly two, components C is selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   and one or more formulation additives, especially one surfactant.

In another preferred embodiment of the invention the SL formulation comprises as component A) quinclorac mono-[2(2-hydroxyethoxy)-ethyl]ammonium, as component B) benzyl alcohol and glycol, and as component C) at least one herbicide selected from the group consisting of
c1) EPSP synthase inhibitors: glyphosate; and
c2) glutamine synthase inhibitors: bilanaphos (bialaphos), glufosinate and glufosinate-P;

including their agriculturally acceptable salts or derivatives;

particularly preferred selected from the group consisting of
c1) EPSP synthase inhibitors: glyphosate, glyphosate-isopropylammonium, glyposate-potassium and glyphosate-trimesium (sulfosate); and
c2) glutamine synthase inhibitors: bilanaphos (bialaphos), bilanaphos-sodium, glufosinate, glufosinate-P, glufosinate-ammonium and glufosinate-P-ammonium;
   especially preferred selected from the group consisting of
c1) EPSP synthase inhibitors: glyphosate, glyphosate-isopropylammonium and glyphosate-trimesium (sulfosate); and
c2) glutamine synthase inhibitors: glufosinate, glufosinate-P, glufosinate-ammonium and glufosinate-P-ammonium;
   more preferred selected from the group consisting of glyphosate-isopropylammonium, glyphosate-trimesium (sulfosate), glufosinate-ammonium and glufosinate-P-ammonium;
   most preferred selected from the group consisting of glyphosate-isopropylammonium and glufosinate-ammonium;
and one or more formulation additives, especially one surfactant.

In another preferred embodiment of the invention the SL formulation comprises as component A) quinclorac mono-[2(2-hydroxyethoxy)-ethyl]ammonium, as component B) benzyl alcohol and glycol, and at least one, preferably exactly one component C selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium;
   and one or more formulation additives, especially one surfactant.

In another preferred embodiment of the invention the SL formulation comprises as component A) quinclorac mono-[2(2-hydroxyethoxy)-ethyl]ammonium, as component B) benzyl alcohol and glycol, and at least one, preferably exactly one component C selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   and one or more formulation additives, especially one surfactant.

In another preferred embodiment of the invention the SL formulation comprises as component A) quinclorac mono-[2(2-hydroxyethoxy)-ethyl]ammonium, as component B) benzyl alcohol and glycol, and at least two, preferably exactly two, components C different from each other, wherein at least one component C is selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium;
   and one or more formulation additives, especially one surfactant.

In another preferred embodiment of the invention the SL formulation comprises as component A) quinclorac mono-[2(2-hydroxyethoxy)-ethyl]ammonium, as component B) benzyl alcohol and glycol, and at least two, preferably exactly two, components C different from each other, wherein at least one component C is selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
   more preferred selected from glufosinate-ammonium and glufosinate-P-ammonium;
most preferred selected from glufosinate-ammonium;
   and one or more formulation additives, especially one surfactant.

In another preferred embodiment of the invention the SL formulation comprises as component A) quinclorac mono-[2(2-hydroxyethoxy)-ethyl]ammonium, as component B) benzyl alcohol and glycol, and at least two, preferably exactly two, components C different from each other, wherein

the first of the two or more, preferably exactly two, components C is selected from glyphosate including its agriculturally acceptable salts or derivatives;
particularly preferred glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium; and
   the second of the two or more, preferably exactly two, components C is selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   and one or more formulation additives, especially one surfactant.

The SL formulation of the invention may also comprise in addition to in addition to components A), B) and C) as defined above, preferably as defined above as preferred, one co-solvent as defined above, especially water, and one or more formulation additives as defined above, especially one surfactant.

In another preferred embodiment of the invention the SL formulation comprises in addition to components A) and B) as defined above, preferably as defined above as preferred, at least one component C) selected from the group consisting of
c1) EPSP synthase inhibitors: glyphosate; and
c2) glutamine synthase inhibitors: bilanaphos (bialaphos), glufosinate and glufosinate-P;

including their agriculturally acceptable salts or derivatives;

particularly preferred selected from the group consisting of
c1) EPSP synthase inhibitors: glyphosate, glyphosate-isopropylammonium, glyposate-potassium and glyphosate-trimesium (sulfosate); and
c2) glutamine synthase inhibitors: bilanaphos (bialaphos), bilanaphos-sodium, glufosinate, glufosinate-P, glufosinate-ammonium and glufosinate-P-ammonium;
   especially preferred selected from the group consisting of
c1) EPSP synthase inhibitors: glyphosate, glyphosate-isopropylammonium and glyphosate-trimesium (sulfosate); and
c2) glutamine synthase inhibitors: glufosinate, glufosinate-P, glufosinate-ammonium and glufosinate-P-ammonium;
   more preferred selected from the group consisting of glyphosate-isopropylammonium, glyphosate-trimesium (sulfosate), glufosinate-ammonium and glufosinate-P-ammonium;
   most preferred selected from the group consisting of glyphosate-isopropylammonium and glufosinate-ammonium;
   one co-solvent, especially water; and
   one or more formulation additives, especially one surfactant.

In another preferred embodiment of the invention the SL formulation comprises as component A) quinclorac dimethylammonium, as component B) benzyl alcohol, and as component C) at least one herbicide selected from the group consisting of
c1) EPSP synthase inhibitors: glyphosate; and
c2) glutamine synthase inhibitors: bilanaphos (bialaphos), glufosinate and glufosinate-P;

including their agriculturally acceptable salts or derivatives;

particularly preferred selected from the group consisting of
c1) EPSP synthase inhibitors: glyphosate, glyphosate-isopropylammonium, glyposate-potassium and glyphosate-trimesium (sulfosate); and
c2) glutamine synthase inhibitors: bilanaphos (bialaphos), bilanaphos-sodium, glufosinate, glufosinate-P, glufosinate-ammonium and glufosinate-P-ammonium;
   especially preferred selected from the group consisting of
c1) EPSP synthase inhibitors: glyphosate, glyphosate-isopropylammonium and glyphosate-trimesium (sulfosate); and
c2) glutamine synthase inhibitors: glufosinate, glufosinate-P, glufosinate-ammonium and glufosinate-P-ammonium;
   more preferred selected from the group consisting of glyphosate-isopropylammonium, glyphosate-trimesium (sulfosate), glufosinate-ammonium and glufosinate-P-ammonium;
   most preferred selected from the group consisting of glyphosate-isopropylammonium and glufosinate-ammonium;
   one co-solvent, especially water; and
   one or more formulation additives, especially one surfactant.

In another preferred embodiment of the invention the SL formulation comprises as component A) quinclorac dimethylammonium, as component B) benzyl alcohol, and at least one, preferably exactly one component C selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium;
   one co-solvent, especially water; and
   one or more formulation additives, especially one surfactant.

In another preferred embodiment of the invention the SL formulation comprises as component A) quinclorac dimethylammonium, as component B) benzyl alcohol, and at least one, preferably exactly one component C selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   one co-solvent, especially water; and
   one or more formulation additives, especially one surfactant.

In another preferred embodiment of the invention the SL formulation comprises as component A) quinclorac dimethylammonium, as component B) benzyl alcohol, and at least two, preferably exactly two, components C different from each other, wherein at least one component C is selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium;
   one co-solvent, especially water; and
   one or more formulation additives, especially one surfactant.

In another preferred embodiment of the invention the SL formulation comprises as component A) quinclorac dimethylammonium, as component B) benzyl alcohol, and at least two, preferably exactly two, components C different from each other, wherein the first of the two or more, preferably exactly two, components C is selected from glyphosate including its agriculturally acceptable salts or derivatives;
particularly preferred glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium; and
   the second of the two or more, preferably exactly two, components C is selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   one co-solvent, especially water; and
   one or more formulation additives, especially one surfactant.

In another preferred embodiment of the invention the SL formulation comprises as component A) quinclorac dimethylammonium, as component B) glycol, and as component C) at least one herbicide selected from the group consisting of
c1) EPSP synthase inhibitors: glyphosate; and
c2) glutamine synthase inhibitors: bilanaphos (bialaphos), glufosinate and glufosinate-P;

including their agriculturally acceptable salts or derivatives;

particularly preferred selected from the group consisting of
c1) EPSP synthase inhibitors: glyphosate, glyphosate-isopropylammonium, glyposate-potassium and glyphosate-trimesium (sulfosate); and
c2) glutamine synthase inhibitors: bilanaphos (bialaphos), bilanaphos-sodium, glufosinate, glufosinate-P, glufosinate-ammonium and glufosinate-P-ammonium;
   especially preferred selected from the group consisting of
c1) EPSP synthase inhibitors: glyphosate, glyphosate-isopropylammonium and glyphosate-trimesium (sulfosate); and
c2) glutamine synthase inhibitors: glufosinate, glufosinate-P, glufosinate-ammonium and glufosinate-P-ammonium;
   more preferred selected from the group consisting of glyphosate-isopropylammonium, glyphosate-trimesium (sulfosate), glufosinate-ammonium and glufosinate-P-ammonium;
   most preferred selected from the group consisting of glyphosate-isopropylammonium and glufosinate-ammonium;
   one co-solvent, especially water; and
   one or more formulation additives, especially one surfactant.

In another preferred embodiment of the invention the SL formulation comprises as component A) quinclorac dimethylammonium, as component B) glycol, and at least one, preferably exactly one component C selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium;
   one co-solvent, especially water; and
   one or more formulation additives, especially one surfactant.

In another preferred embodiment of the invention the SL formulation comprises as component A) quinclorac dimethylammonium, as component B) glycol, and at least one, preferably exactly one component C selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   one co-solvent, especially water; and
   one or more formulation additives, especially one surfactant.

In another preferred embodiment of the invention the SL formulation comprises as component A) quinclorac dimethylammonium, as component B) glycol, and at least two, preferably exactly two, components C different from each other, wherein at least one component C is selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium;
   one co-solvent, especially water; and
   one or more formulation additives, especially one surfactant.

In another preferred embodiment of the invention the SL formulation comprises as component A) quinclorac dimethylammonium, as component B) glycol, and at least two, preferably exactly two, components C different from each other, wherein at least one component C is selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   one co-solvent, especially water; and
   one or more formulation additives, especially one surfactant.

In another preferred embodiment of the invention the SL formulation comprises as component A) quinclorac dimethylammonium, as component B) glycol, and at least two, preferably exactly two, components C different from each other, wherein the first of the two or more, preferably exactly two, components C is selected from glyphosate including its agriculturally acceptable salts or derivatives;
particularly preferred glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium; and
   the second of the two or more, preferably exactly two, components C is selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   one co-solvent, especially water; and
   one or more formulation additives, especially one surfactant.

In another preferred embodiment of the invention the SL formulation comprises as component A) quinclorac dimethylammonium, as component B) benzyl alcohol and glycol, and as component C) at least one herbicide selected from the group consisting of
c1) EPSP synthase inhibitors: glyphosate; and
c2) glutamine synthase inhibitors: bilanaphos (bialaphos), glufosinate and glufosinate-P;

including their agriculturally acceptable salts or derivatives;

particularly preferred selected from the group consisting of
c1) EPSP synthase inhibitors: glyphosate, glyphosate-isopropylammonium, glyposate-potassium and glyphosate-trimesium (sulfosate); and
c2) glutamine synthase inhibitors: bilanaphos (bialaphos), bilanaphos-sodium, glufosinate, glufosinate-P, glufosinate-ammonium and glufosinate-P-ammonium;
   especially preferred selected from the group consisting of
c1) EPSP synthase inhibitors: glyphosate, glyphosate-isopropylammonium and glyphosate-trimesium (sulfosate); and
c2) glutamine synthase inhibitors: glufosinate, glufosinate-P, glufosinate-ammonium and glufosinate-P-ammonium;
   more preferred selected from the group consisting of glyphosate-isopropylammonium, glyphosate-trimesium (sulfosate), glufosinate-ammonium and glufosinate-P-ammonium;
   most preferred selected from the group consisting of glyphosate-isopropylammonium and glufosinate-ammonium;
   one co-solvent, especially water; and
   one or more formulation additives, especially one surfactant.

In another preferred embodiment of the invention the SL formulation comprises as component A) quinclorac dimethylammonium, as component B) benzyl alcohol and glycol, and at least one, preferably exactly one component C selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium;
   one co-solvent, especially water; and
   one or more formulation additives, especially one surfactant.

In another preferred embodiment of the invention the SL formulation comprises as component A) quinclorac dimethylammonium, as component B) benzyl alcohol and glycol, and at least one, preferably exactly one component C selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   one co-solvent, especially water; and
   one or more formulation additives, especially one surfactant.

In another preferred embodiment of the invention the SL formulation comprises as component A) quinclorac dimethylammonium, as component B) benzyl alcohol and glycol, and at least two, preferably exactly two, components C different from each other, wherein at least one component C is selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium;
   one co-solvent, especially water; and
   one or more formulation additives, especially one surfactant.

In another preferred embodiment of the invention the SL formulation comprises as component A) quinclorac dimethylammonium, as component B) benzyl alcohol and glycol, and at least two, preferably exactly two, components C different from each other, wherein at least one component C is selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
   more preferred selected from glufosinate-ammonium and glufosinate-P-ammonium;
   most preferred selected from glufosinate-ammonium;
   one co-solvent, especially water; and
   one or more formulation additives, especially one surfactant.

In another preferred embodiment of the invention the SL formulation comprises as component A) quinclorac dimethylammonium, as component B) benzyl alcohol and glycol, and at least two, preferably exactly two, components C different from each other, wherein

the first of the two or more, preferably exactly two, components C is selected from glyphosate including its agriculturally acceptable salts or derivatives;
particularly preferred glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium; and
   the second of the two or more, preferably exactly two, components C is selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   one co-solvent, especially water; and
   one or more formulation additives, especially one surfactant.

In another preferred embodiment of the invention the SL formulation comprises as component A) quinclorac mono-[2(2-hydroxyethoxy)-ethyl]ammonium, as component B) benzyl alcohol, and as component C) at least one herbicide selected from the group consisting of
c1) EPSP synthase inhibitors: glyphosate; and
c2) glutamine synthase inhibitors: bilanaphos (bialaphos), glufosinate and glufosinate-P;

including their agriculturally acceptable salts or derivatives;

particularly preferred selected from the group consisting of
c1) EPSP synthase inhibitors: glyphosate, glyphosate-isopropylammonium, glyposate-potassium and glyphosate-trimesium (sulfosate); and
c2) glutamine synthase inhibitors: bilanaphos (bialaphos), bilanaphos-sodium, glufosinate, glufosinate-P, glufosinate-ammonium and glufosinate-P-ammonium;
   especially preferred selected from the group consisting of
c1) EPSP synthase inhibitors: glyphosate, glyphosate-isopropylammonium and glyphosate-trimesium (sulfosate); and
c2) glutamine synthase inhibitors: glufosinate, glufosinate-P, glufosinate-ammonium and glufosinate-P-ammonium;
   more preferred selected from the group consisting of glyphosate-isopropylammonium, glyphosate-trimesium (sulfosate), glufosinate-ammonium and glufosinate-P-ammonium;
   most preferred selected from the group consisting of glyphosate-isopropylammonium and glufosinate-ammonium;
   one co-solvent, especially water; and
   one or more formulation additives, especially one surfactant.

In another preferred embodiment of the invention the SL formulation comprises as component A) quinclorac mono-[2(2-hydroxyethoxy)-ethyl]ammonium, as component B) benzyl alcohol, and at least one, preferably exactly one component C selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium;
   one co-solvent, especially water; and
   one or more formulation additives, especially one surfactant.

In another preferred embodiment of the invention the SL formulation comprises as component A) quinclorac mono-[2(2-hydroxyethoxy)-ethyl]ammonium, as component B) benzyl alcohol, and at least one, preferably exactly one component C selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   one co-solvent, especially water; and
   one or more formulation additives, especially one surfactant.

In another preferred embodiment of the invention the SL formulation comprises as component A) quinclorac mono-[2(2-hydroxyethoxy)-ethyl]ammonium, as component B) benzyl alcohol, and at least two, preferably exactly two, components C different from each other, wherein at least one component C is selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium;
   one co-solvent, especially water; and
   one or more formulation additives, especially one surfactant.

In another preferred embodiment of the invention the SL formulation comprises as component A) quinclorac mono-[2(2-hydroxyethoxy)-ethyl]ammonium, as component B) benzyl alcohol, and at least two, preferably exactly two, components C different from each other, wherein at least one component C is selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   one co-solvent, especially water; and
   one or more formulation additives, especially one surfactant.

In another preferred embodiment of the invention the SL formulation comprises as component A) quinclorac mono-[2(2-hydroxyethoxy)-ethyl]ammonium, as component B) benzyl alcohol, and at least two, preferably exactly two, components C different from each other, wherein

the first of the two or more, preferably exactly two, components C is selected from glyphosate including its agriculturally acceptable salts or derivatives;
particularly preferred glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium; and
   the second of the two or more, preferably exactly two, components C is selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   one co-solvent, especially water; and
   one or more formulation additives, especially one surfactant.

In another preferred embodiment of the invention the SL formulation comprises as component A) quinclorac mono-[2(2-hydroxyethoxy)-ethyl]ammonium, as component B) glycol, and as component C) at least one herbicide selected from the group consisting of c1) EPSP synthase inhibitors: glyphosate; and
c2) glutamine synthase inhibitors: bilanaphos (bialaphos), glufosinate and glufosinate-P;

including their agriculturally acceptable salts or derivatives;

particularly preferred selected from the group consisting of
c1) EPSP synthase inhibitors: glyphosate, glyphosate-isopropylammonium, glyposate-potassium and glyphosate-trimesium (sulfosate); and
c2) glutamine synthase inhibitors: bilanaphos (bialaphos), bilanaphos-sodium, glufosinate, glufosinate-P, glufosinate-ammonium and glufosinate-P-ammonium;
   especially preferred selected from the group consisting of
c1) EPSP synthase inhibitors: glyphosate, glyphosate-isopropylammonium and glyphosate-trimesium (sulfosate); and
c2) glutamine synthase inhibitors: glufosinate, glufosinate-P, glufosinate-ammonium and glufosinate-P-ammonium;
   more preferred selected from the group consisting of glyphosate-isopropylammonium, glyphosate-trimesium (sulfosate), glufosinate-ammonium and glufosinate-P-ammonium;
   most preferred selected from the group consisting of glyphosate-isopropylammonium and glufosinate-ammonium;
   one co-solvent, especially water; and
   one or more formulation additives, especially one surfactant.

In another preferred embodiment of the invention the SL formulation comprises as component A) quinclorac mono-[2(2-hydroxyethoxy)-ethyl]ammonium, as component B) glycol, and at least one, preferably exactly one component C selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium;
   one co-solvent, especially water; and
   one or more formulation additives, especially one surfactant.

In another preferred embodiment of the invention the SL formulation comprises as component A) quinclorac mono-[2(2-hydroxyethoxy)-ethyl]ammonium, as component B) glycol, and at least one, preferably exactly one component C selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   one co-solvent, especially water; and
   one or more formulation additives, especially one surfactant.

In another preferred embodiment of the invention the SL formulation comprises as component A) quinclorac mono-[2(2-hydroxyethoxy)-ethyl]ammonium, as component B) glycol, and at least two, preferably exactly two, components C different from each other, wherein at least one component C is selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium;
   one co-solvent, especially water; and
   one or more formulation additives, especially one surfactant.

In another preferred embodiment of the invention the SL formulation comprises as component A) quinclorac mono-[2(2-hydroxyethoxy)-ethyl]ammonium, as component B) glycol, and at least two, preferably exactly two, components C different from each other, wherein at least one component C is selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   one co-solvent, especially water; and
   one or more formulation additives, especially one surfactant.

In another preferred embodiment of the invention the SL formulation comprises as component A) quinclorac mono-[2(2-hydroxyethoxy)-ethyl]ammonium, as component B) glycol, and at least two, preferably exactly two, components C different from each other, wherein

the first of the two or more, preferably exactly two, components C is selected from glyphosate including its agriculturally acceptable salts or derivatives;
particularly preferred glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium; and
   the second of the two or more, preferably exactly two, components C is selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   one co-solvent, especially water; and
   one or more formulation additives, especially one surfactant.

In another preferred embodiment of the invention the SL formulation comprises as component A) quinclorac mono-[2(2-hydroxyethoxy)-ethyl]ammonium, as component B) benzyl alcohol and glycol, and as component C) at least one herbicide selected from the group consisting of
c1) EPSP synthase inhibitors: glyphosate; and
c2) glutamine synthase inhibitors: bilanaphos (bialaphos), glufosinate and glufosinate-P;
including their agriculturally acceptable salts or derivatives;
   particularly preferred selected from the group consisting of
c1) EPSP synthase inhibitors: glyphosate, glyphosate-isopropylammonium, glyposate-potassium and glyphosate-trimesium (sulfosate); and
c2) glutamine synthase inhibitors: bilanaphos (bialaphos), bilanaphos-sodium, glufosinate, glufosinate-P, glufosinate-ammonium and glufosinate-P-ammonium;
   especially preferred selected from the group consisting of
c1) EPSP synthase inhibitors: glyphosate, glyphosate-isopropylammonium and glyphosate-trimesium (sulfosate); and
c2) glutamine synthase inhibitors: glufosinate, glufosinate-P, glufosinate-ammonium and glufosinate-P-ammonium;
   more preferred selected from the group consisting of glyphosate-isopropylammonium, glyphosate-trimesium (sulfosate), glufosinate-ammonium and glufosinate-P-ammonium;
   most preferred selected from the group consisting of glyphosate-isopropylammonium and glufosinate-ammonium;
   one co-solvent, especially water; and
   one or more formulation additives, especially one surfactant.

In another preferred embodiment of the invention the SL formulation comprises as component A) quinclorac mono-[2(2-hydroxyethoxy)-ethyl]ammonium, as component B) benzyl alcohol and glycol, and at least one, preferably exactly one component C selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium;
   one co-solvent, especially water; and
   one or more formulation additives, especially one surfactant.

In another preferred embodiment of the invention the SL formulation comprises as component A) quinclorac mono-[2(2-hydroxyethoxy)-ethyl]ammonium, as component B) benzyl alcohol and glycol, and at least one, preferably exactly one component C selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   one co-solvent, especially water; and
   one or more formulation additives, especially one surfactant.

In another preferred embodiment of the invention the SL formulation comprises as component A) quinclorac mono-[2(2-hydroxyethoxy)-ethyl]ammonium, as component B) benzyl alcohol and glycol, and at least two, preferably exactly two, components C different from each other, wherein at least one component C is selected from the group of c1): EPSP synthase inhibitors including their agriculturally acceptable salts or derivatives;
preferably selected from glyphosate including its agriculturally acceptable salts or derivatives;
particularly preferred selected from glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium;
   one co-solvent, especially water; and
   one or more formulation additives, especially one surfactant.

In another preferred embodiment of the invention the SL formulation comprises as component A) quinclorac mono-[2(2-hydroxyethoxy)-ethyl]ammonium, as component B) benzyl alcohol and glycol, and at least two, preferably exactly two, components C different from each other, wherein at least one component C is selected from the group of c2): glutamine synthase inhibitors including their agriculturally acceptable salts or derivatives;
preferably selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
   most preferred selected from glufosinate-ammonium;
   one co-solvent, especially water; and
   one or more formulation additives, especially one surfactant.

In another preferred embodiment of the invention the SL formulation comprises as component A) quinclorac mono-[2(2-hydroxyethoxy)-ethyl]ammonium, as component B) benzyl alcohol and glycol, and at least two, preferably exactly two, components C different from each other, wherein

the first of the two or more, preferably exactly two, components C is selected from glyphosate including its agriculturally acceptable salts or derivatives;
particularly preferred glyphosate, glyphosate-isopropylammonium, glyposate-potassium and glyphosate-trimesium (sulfosate);
especially preferred selected from glyphosate, glyphosate-isopropylammonium and glyphosate-trimesium (sulfosate);
more preferred selected from glyphosate-isopropylammonium and glyphosate-trimesium (sulfosate);
most preferred selected from glyphosate-isopropylammonium; and
   the second of the two or more, preferably exactly two, components C is selected from bilanaphos (bialaphos), glufosinate and glufosinate-P, including their agriculturally acceptable salts or derivatives;
particularly preferred selected from bilanaphos (bialaphos), bilanaphos-sodium, glufosinate, glufosinate-P, glufosinate-ammonium and glufosinate-P-ammonium;
especially preferred selected from glufosinate, glufosinate-P, glufosinate-ammonium and glufosinate-P-ammonium;
more preferred selected from glufosinate-ammonium and glufosinate-P-ammonium;
most preferred selected from glufosinate-ammonium;
   one co-solvent, especially water; and
   one or more formulation additives, especially one surfactant.

The amount of the quinclorac ammonium salt of formula I in the SL formulation of the invention usually ranges from 1 to 40% by weight, preferably from 5 to 30% by weight and in particular from 10 to 20% by weight based on the total weight of said formulation.

The amount of the solvent of formula IIa and/or IIb in the SL formulation of the invention usually ranges from 10 to 95% by weight, preferably from 20 to 70% by weight and in particular from 30 to 60% by weight based on the total weight of said formulation.

The SL formulation of the present invention may also comprise one or more additional herbicidal active ingredients, especially in an amount from 1 to 40% by weight, preferably from 2 to 30% by weight and in particular from 2 to 25% by weight (based on the total weight of said formulation).

The SL formulation of the present invention may also comprise one or more co-solvents, especially in an amount from 10 to 90% by weight, preferably from 30 to 60% by weight and in particular from 40 to 50% by weight based on the total weight of said formulation.

The SL formulation of the present invention may also comprise one or more formulation additives, especially in an amount from 0.1 to 20% by weight based on the total weight of said formulation.

The SL formulation according to the present invention can be prepared by adding the quinclorac ammonium salt of formula I and the additional herbicidal active ingredient(s) C, co-solvent(s) and if desired the formulation additive(s) to the solvent of formula IIa and/or IIb, while stirring, and, optionally while heating.

It is also possible that some of the components of the SL formulation are premixed and the remaining components are added thereafter while stirring, and, optionally while heating.

In another embodiment the SL formulation can be prepared by mixing together quinclorac “acid”, an amine of formula III

NR¹R²R³ (III)

wherein the meaning of R¹, R² and R³ are those given for the quinclorac ammonium salt of formula I,
and, if desired one or more additional herbicidal active ingredients and/one or more solvents and/or one or more formulation additives, wherein the amounts of quinclorac “acid” and of the amine of formula III are from 0.8:1 to 1.2:1 mol %, and the amounts of other components are as desired.

Especially quinclorac “acid” and the amine of formula III are used in equimolar amounts.

In case one of the additional herbicidal active ingredients is also used in its [NR₁R₂R₃H]⁺ form the respective “neutral form” of the additional herbicidal active ingredient may be mixed together with the other components as mentioned above and an additional respective amount of the amine of formula III, which is in the range of from 0.8:1 to 1.2:1 mol %.

In a preferred embodiment the additional herbicidal active ingredient and the additional amount of amine of formula III are used in an equimolar ratio.

In a special embodiment the amine of formula III is used as aqueous solution.

If desired, the additional herbicidal active ingredients and the formulation additives may be contained within the SL formulation of the present invention. However, it is also possible to add these components after dilution with water to the ready-to-use aqueous composition.

Upon dilution with water, the SL formulation of the invention forms an aqueous herbicide composition which comprises a quinclorac ammonium salt of formula I, a solvent of formula IIa and/or IIb, water and optionally one or more additional herbicidal active ingredients, and/or one or more co-solvents and/or one or more formulation additives.

In order to obtain these aqueous herbicide compositions, the SL formulation of the invention are usually diluted with at least 1.parts of water, preferably at least 20 parts of water, in particular at least 40 parts of water and more preferably at least 80 parts of water (all parts are given in parts by weight) (based on the total weight of the aqueous herbicide compositions).

Dilution will be usually achieved by pouring the SL formulations of the invention into water. Usually, dilution is achieved with agitation, e.g. with stirring, to ensure a rapid mixing of the concentrate in water. However, agitation is not necessary. Though the temperature of mixing is not critical, mixing is usually performed at temperatures ranging from 0 to 100° C., in particular from 10 to 50° C. or at ambient temperature.

The water used for mixing is usually tap water. However the water may already contain further compounds which are used in plant protection, e.g. nutrificants, fertilizers, water soluble pesticides or alkylates of vetetable oils, such as methylated seed oil (MSO), preferably MSO. It is also possible that the SL formulation is mixed with water and if desired one or more of said further compounds which are used in plant protection, e.g. nutrificants, fertilizers, water soluble pesticides or alkylates of vetetable oils, such as methylated seed oil (MSO), are added.

If desired, MSO is employed in amounts from 0.125 to 5% by volume, preferably from 0.25 to 2.5% by volume and in particular from 0.5 to 1% by volume (based on the total volume of the aqueous herbicide compositions=final volume for application).

The aqueous herbicide compositions of the invention can be used as such to control undesired vegetation. Therefore, the present invention also relates to an aqueous herbicide composition which is obtained by diluting the SL formulation of the present invention with water and optionally with further compounds which are used in plant protection, e.g. nutrificants, fertilizers, water soluble pesticides or alkylates of vetetable oils, such as methylated seed oil (MSO).

In a preferred embodiment of the aqueous herbicide compositions the SL formulation is diluted with water only.

In a further preferred embodiment of the aqueous herbicide compositions the SL formulation is diluted with water and alkylates of vetetable oils, especially MSO.

In a further preferred embodiment of the aqueous herbicide compositions the SL formulation is diluted with water and alkylates of vetetable oils, especially MSO, only.

The present invention also relates to the use of said aqueous herbicide compositions for control of undesired vegetation by contacting the undesired plants, their habitat and/or their seeds with an effective amount of an aqueous herbicidal composition as described herein.

The compositions of the invention after dilution are applied by usual means which are familiar to a skilled person.

The present invention also relates to herbicidal active mixtures comprising at least one quinclorac ammonium salt of formula I as described herein and at least one further herbicide C selected from the group consisting of c1) to c2):
c1) enolpyruvyl shikimate 3-phosphate synthase inhibitors (EPSP inhibitors); and
c2) glutamine synthetase inhibitors;
   including their agriculturally acceptable salts or derivatives,
   as described above.

With regard to the preferred embodiments of the quinclorac ammonium salt of formula I, the preferrencies as mentioned hereinbefore are fully applicable.

With regard to the preferred embodiments of the herbicide C, the preferrencies as mentioned hereinbefore are fully applicable.

The compounds of formula I can be prepared by mixing together quinclorac “acid” and an amine of formula III

NR¹R²R³ (III)

wherein the meaning of R¹, R² and R³ are those given for the quinclorac ammonium salt of formula I. The reaction may be performed in substance or in an appropriate sok vent, for example water, an alcohol, like methanol and ethanol, a ketone, like acetone, diethylketone.

The herbicides C of groups c1) to and c2) are known herbicides, see, for example, The Compendium of Pesticide Common Names (http://www.alanwood.net/pesticides/).

According to one embodiment, individual components of the herbicidal active mixtures according to the invention such as parts of a kit or parts of a binary mixture may be mixed by the user himself in a spray tank and further auxiliaries may be added, if appropriate.

In a further embodiment, either individual components of the herbicidal active mixtures according to the invention or partially premixed components, e.g. agrochemical components comprising compounds I and/or active substances from the group C may be mixed by the user in a spray tank and further auxiliaries and additives may be added, if appropriate.

In a further embodiment, either individual components of the herbicidal active mixtures according to the invention or partially premixed components, e.g. components comprising compounds I and/or active substances from the group C, can be applied jointly (e.g. after tank mix) or consecutively.

The active compound A and the at least one further active compound C can be formulated and applied jointly or separately, simultaneously or in succession, before, during or after the emergence of the plants. In case of separate application, the order of the application of the active compounds A and C is of minor importance. The only thing that is important is that the at least one active compound A and the at least one further active compound C are present simultaneously at the site of action, i.e. are at the same time in contact with or taken up by the plant to be controlled.

In the case of separate application it is of minor importance, in which order the application takes place. It is only necessary, that the herbicide compound A and the herbicide compound C are applied in a time frame that allows simultaneous action of the active ingredients on the plants, preferably within a time-frame of at most 14 days, in particular at most 7 days.

Accordingly, a first embodiment of the invention relates to compositions in the form of a crop protection composition formulated as a 1-component composition comprising the at least one active compound of formula I or the at least one active compound of formula I (active compound A) and at least one further active compound selected from the herbicides C.

Accordingly, a second embodiment of the invention relates to compositions in the form of a crop protection composition formulated as a 2-component composition comprising a first formulation (component) comprising the at least one active compound A, and a second component comprising at least one further active compound selected from the herbicides C.

Usually the application rate of the quinclorac ammonium salt of formula I, calculated on the basis of the quinclorac “acid”, is in the range of 20 to 1700 g ai/ha, preferably 20 to 500 g ai/ha, particularly preferred 20 to 200 g ai/ha, especially preferred 50 to 200 g ai/ha.

Usually the application rate of glyphosate, its salts, esters and/or amids, calculated on the basis of the glyphosate “acid”, is in the range of 300 to 1000 g ai/ha, preferably 450 to 900 g ai/ha, particularly preferred 450 to 600 g ai/ha.

Usually the application rate of bilanaphos (bialaphos), its salts, esters and/or amids, calculated on the basis of the bilanaphos (bialaphos) “acid” is in the range of 300 to 1000 g ai/ha, preferably 450 to 900 g ai/ha, particularly preferred 450 to 600 g ai/ha.

Usually the application rate of glufosinate or glufosinate-P, and their salts, esters and/or amids, calculated on the basis of the glufosinate “acid” and the glufosinate P “acid” respectively, is in the range of 300 to 1000 g ai/ha, preferably 450 to 900 g ai/ha, particularly preferred 500 to 600 g ai/ha.

A SL formulation represents a liquid, homogeneous formulation. Usually the active ingredient(s) of the SL formulation is/are dissolved in water or a combination of water and suitable water soluble solvent(s) plus optionally other ingredients. The dilution of a SL formulation with water results in a clear solution.

The herbicidal active mixtures and formulations according to the present invention have an improved efficacy against undesired weeds, preferably against small seeded broadleaf weed species, more preferably against Galium aparine.

The SL formulation and the compositions according to the present invention are are highly active against unwanted harmful plants in crops, preferably in brassica crop species, more preferably in rapeseed cultures, particularly preferred in oilseed rape and canola. Thus they are especially useful in plant protection of rapeseed. The term “plant” as used herein includes all parts of a plant such as germinating seeds, emerging seedlings and herbaceous vegetation including all belowground portions (such as the roots) and aboveground portions.

Rapeseed (Brassica napus) is also known as rape, oilseed rape, rapa, rappi, rapeseed, colza and, in the case of one particular group of cultivars, canola. In the terms of the present invention, “rapeseed” is used as a synonym for all these terms, inclusive canola, as well as other cultivated Brassica species, such as for example field mustard (Brassica campestris) or Indian mustard (Brassica juncea).

The SL formulation and the compositions according to the invention can also be used in genetically modified rapeseed plants. The term “genetically modified plants” is to be understood as plants whose genetic material has been modified by the use of recombinant DNA techniques to include an inserted sequence of DNA that is not native to that plant species' genome or to exhibit a deletion of DNA that was native to that species' genome, wherein the modification(s) cannot readily be obtained by cross breeding, mutagenesis or natural recombination alone. Often, a particular genetically modified plant will be one that has obtained its genetic modification(s) by inheritance through a natural breeding or propagation process from an ancestral plant whose genome was the one directly treated by use of a recombinant DNA technique. Typically, one or more genes have been integrated into the genetic material of a genetically modified plant in order to improve certain properties of the plant. Such genetic modifications also include but are not limited to targeted post-translational modification of protein(s), oligo- or polypeptides. e.g., by inclusion therein of amino acid mutation(s) that permit, decrease, or promote glycosylation or polymer additions such as prenylation, acetylation farnesylation, or PEG moiety attachment.

Rapeseed plants as well as the propagation material of said plants, which can be treated with the inventive mixtures include all modified non-transgenic plants or transgenic plants, e.g. crops which tolerate the action of herbicides or fungicides or insecticides owing to breeding, including genetic engineering methods, or plants which have modified characteristics in comparison with existing plants, which can be generated for example by traditional breeding methods and/or the generation of mutants, or by recombinant procedures.

For example, the SL formulation and the compositions according to the present invention can be applied (as seed treatment, foliar spray treatment, in-furrow application or by any other means) also to plants which have been modified by breeding, mutagenesis or genetic engineering including but not limiting to agricultural biotech products on the market or in development (cf. http://www.bio.org/speeches/pubs/er/agri_products.asp).

Rapeseed plants that have been modified by breeding, mutagenesis or genetic engineering, e.g. have been rendered tolerant to applications of specific classes of herbicides, such as auxinic herbicides such as dicamba or 2,4-D; bleacher herbicides such as 4-hydroxyphenylpyruvate dioxygenase (HPPD) inhibitors or phytoene desaturase (PDS) inhibitors; acetolactate synthase (ALS) inhibitors such as sulfonylureas or imidazolinones, e.g. imazamox; enolpyruvyl shikimate 3-phosphate synthase (EPSP) inhibitors such as glyphosate or sulfosate; glutamine synthetase (GS) inhibitors such as glufosinate or bialafos; protoporphyrinogen-IX oxidase (PPO) inhibitors; lipid biosynthesis inhibitors such as acetylCoA carboxylase (ACCase) inhibitors; or photosynthetic electron transport inhibitors at the photosystem II receptor site, such as bromoxynil, ioxynil, atrazine, simazine or terbutylazine as a result of conventional methods of breeding or genetic engineering; furthermore, plants have been made resistant to multiple classes of herbicides through multiple genetic modifications, such as resistance to both glyphosate and glufosinate or to both glyphosate and a herbicide from another class such as ALS inhibitors, HPPD inhibitors, auxinic herbicides, or ACCase inhibitors. These herbicide resistance technologies are, for example, described in Pest Management Science 61, 2005, 246; 61, 2005, 258; 61, 2005, 277; 61, 2005, 269; 61, 2005, 286; 64, 2008, 326; 64, 2008, 332; Weed Science 57, 2009, 108; Australian Journal of Agricultural Research 58, 2007, 708; Science 316, 2007, 1185; and references quoted therein. Several cultivated plants have been rendered tolerant to herbicides by mutgenesis and conventional methods of breeding, e.g., Clearfield® summer rape (Canola, BASF SE, Germany) being tolerant to imidazolinones, e.g., imazamox. Genetic engineering methods have been used to render rapeseed plants tolerant to herbicides such as glyphosate, imidazolinones and glufosinate, some of which are under development or commercially available under the brands or trade names RoundupReady® (glyphosate tolerant, Monsanto, USA) and LibertyLink® (glufosinate tolerant, Bayer CropScience, Germany). Preferably, the rapeseed plants are tolerant against herbicides selected from the group of photosynthetic electron transport inhibitors at the photosystem II receptor site, e.g. atrazine, simazine, terbutylazine or bromoxynil or its agriculturally acceptable esters; acetolactate synthase inhibitors (ALS inhibitors), e.g. imazamox or its agriculturally acceptable salts; auxinic herbicides, e.g. 2,4-D, dicamba and their agriculturally acceptable salts, esters and amides; EPSP synthase inhibitors, e.g. glyphosate, sulfosate and their agriculturally acceptable salts; and glutamine synthase inhibitors, e.g. glufosinate, bialafos and their agriculturally acceptable salts.

Furthermore, rapeseed plants are also covered that are by the use of recombinant DNA techniques capable to synthesize one or more insecticidal proteins, especially those known from the bacterial genus Bacillus, particularly from Bacillus thuringiensis, such as delta-endotoxins, e.g., CryIA(b), CryIA(c), CryIF, CryIF(a2), CryIIA(b), CryIIIA, CryIIIB(b1) or Cry9c; vegetative insecticidal proteins (VIP), e.g., VIP1, VIP2, VIP3 or VIP3A; insecticidal proteins of bacteria colonizing nematodes, e.g., Photorhabdus spp. or Xenorhabdus spp.; toxins produced by animals, such as scorpion toxins, arachnid toxins, wasp toxins, or other insect-specific neurotoxins; toxins produced by fungi, such as Streptomycetes toxins, plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxy-steroid oxidase, ecdysteroid-IDP-glycosyl-transferase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilbene synthase, bibenzyl synthase, chitinases or glucanases. In the context of the present invention these insecticidal proteins or toxins are to be understood expressly also as including pre-toxins, hybrid proteins, truncated or otherwise modified proteins. Hybrid proteins are characterized by a new combination of protein domains, (see, e.g., WO 02/015701). Further examples of such toxins or genetically modified plants capable of synthesizing such toxins are disclosed, e.g., in EP-A 374 753, WO 93/007278, WO 95/34656, EP-A 427 529, EP-A 451 878, WO 03/18810 and WO 03/52073. The methods for producing such genetically modified plants are generally known to the person skilled in the art and are described, e.g., in the publications mentioned above. These insecticidal proteins contained in the genetically modified plants impart to the plants producing these proteins tolerance to harmful pests from all taxonomic groups of arthropods, especially to beetles (Coleoptera), two-winged insects (Diptera), and moths (Lepidoptera) and to nematodes (Nematoda). Genetically modified plants capable to synthesize one or more insecticidal proteins are, e.g., described in the publications mentioned above.

Furthermore, rapeseed plants are also covered that are by the use of recombinant DNA techniques capable to synthesize one or more proteins to increase the resistance or tolerance of those plants to bacterial, viral or fungal pathogens. Examples of such proteins are the so-called “pathogenesis-related proteins” (PR proteins, see, e.g., EP-A 392 225), plant disease resistance genes or T4-lyso-zym. The methods for producing such genetically modified plants are generally known to the person skilled in the art and are described, e.g., in the publications mentioned above.

Furthermore, rapeseed plants are also covered that are by the use of recombinant DNA techniques capable to synthesize one or more proteins to increase the productivity (e.g., bio-mass production, grain yield, starch content, oil content or protein content), tolerance to drought, salinity or other growth-limiting environmental factors or tolerance to pests and fungal, bacterial or viral pathogens of those plants.

Furthermore, plants are also covered that contain by the use of recombinant DNA techniques a modified amount of ingredients or new ingredients, specifically to improve human or animal nutrition, e.g., oil crops that produce health-promoting long-chain omega-3 fatty acids or unsaturated omega-9 fatty acids (e.g., Nexera® rape, Dow AgroSciences, Canada).

Furthermore, plants are also covered that contain by the use of recombinant DNA techniques a modified amount of ingredients or new ingredients, specifically to improve raw material production.

The SL formulation and the compositions to be used according to the invention are suitable as herbicides. They are suitable as such or as an appropriately formulated composition. The compositions according to the invention control vegetation on noncrop areas very efficiently, especially at high rates of application. They act against broad-leafed weeds and grass weeds in rapeseed crops without causing any significant damage to the crop plants. This effect is mainly observed at low rates of application.

The following examples are intended to further illustrate the present invention.

### Biological Tests

Field trials were conducted using tank mixtures of Liberty Link® (LL) herbicide with ACCORD® 75DF or ACCORD® 180SL at the rates specified in Table A below. In all cases MERGE® adjuvant was included as part of the tank mix application.

Applications were made post-emergence using small plot research sprayers with a variety of nozzles ranging from fine to course droplet size and were applied with a water volume of 100 L/ha (unless otherwise noted).

All trials utilized a randomized complete block design with four replications. Since evaluations were made at various intervals not common to all trials, the specific rating intervals are specified in Table A as days after treatment (DAT).

Efficacy for herbicide treatments was evaluated using visual assessments expressed as percent (%) weed control compared to an untreated weedy check. 100 means no emergence of the plants, or complete destruction of at least the above-ground parts, and 0 means no damage or normal course of growth.

TABLE A
GALAP control in Liberty Link Canola by post-emergence
treatment by glufosinate (Liberty Link ®) in combination
with either quinclorac (ACCORD ® 75DF) or
quinclorac ammonium salt (ACCORD ® 180SL).

| | | | Damage of GALAP | |
|---|---|---|---|---|
| | | | 22 to 28 | 43 to 58 |
| | | Rate (g ai/ha/ | days after | days after |
| Example | Treatment | *L/ha) | treatment | treatment |
| Example 1 | LIBERTY ® | 500 + 50 + 1* | 82.6 | 87.2 |
| (comparative) | 150SL + | | | |
| | ACCORD ® | | | |
| | 75DF + | | | |
| | MERGE | | | |
| Example 2 | LIBERTY ® | 500 + 50 + 1* | 88.9 | 90.0 |
| (according to the | 150SL + | | | |
| invention) | ACCORD ® | | | |
| | 180SL + | | | |
| | MERGE ® | | | |

GALAP Galium aparine L. (cleaver)
Liberty Link ® Canola glufosinate-resistant canola
LIBERTY ® 150 SL glufosinate ammonium and adjuvants
ACCORD ® 75 DF quinclorac and adjuvants
ACCORD ® 180 SL quinclorac dimethylammonium and adjuvants
MERGE ® Adjuvant surfactant blend and petroleum hydrocarbons.

These data clearly demonstrate that the treatment by glufosinate in combination with quinclorac ammonium salt provided increased cleaver control over the treatment by glufosinate in combination with quinclorac in form of the free acid.

1. A method for controlling undesirable vegetation, which comprises diluting a herbicidal active amount of a SL formulation with water, and allowing said diluted formulation to act on plants, their habitat and/or their seeds, wherein the soluble liquid (SL) formulation comprises:
A) a quinclorac ammonium salt of formula I 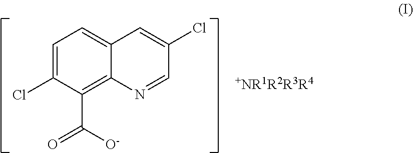
wherein
R¹, R², R³ and R⁴ are independently hydrogen, C₁-C₆-alkyl, —(CHR^{a}—CHR^{b}—Z₁)ₓ—H or —(CHR^{a}—CHR^{b}—CHR^{c}—CHR^{d}—Z₂)_{y}—H (wherein R^{a}, R^{b}, R^{c} and R^{d} are independently hydrogen or C₁-C₆-alkyl, wherein Z₁ and Z₂ are independently O, NH or N—C₁-C₆-alkyl, and wherein x and y are independently an integer from 1 to 6);
B) a solvent of formula IIa 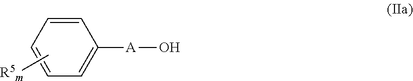
wherein
R⁵ is C₁-C₆-alkyl;
A is C₁-C₆-alkylene or C₁-C₆-oxyalkylene; and
m is 0, 1, 2 or 3;
and/or a solvent of formula IIb
HO—B—OH (IIb)
wherein
B is a straight-chain or branched C₂-C₈-alkylene or C₂-C₄-alkyleneoxy-C₂-C₄-alkylene or C₂-C₃-alkyleneoxy-C₂-C₃-alkyleneoxy-C₂-C₃-alkylene;
and
C) at least one further herbicides selected from the group consisting of c1) to
c2):
c1) enolpyruvyl shikimate 3-phosphate synthase inhibitors (EPSP inhibitors); and
c2) glutamine synthetase inhibitors;
including their agriculturally acceptable salts or derivatives.

2. The method according to claim 1 wherein
R¹ is hydrogen or C₁-C₆-alkyl;
R², R³ are independently C₁-C₆-alkyl;
R⁴ is hydrogen.

3. The method according to claim 1 wherein
R¹, R⁴ are hydrogen;
R², R³ are independently C₁-C₆-alkyl.

4. The method according to claim 1 wherein the quinclorac ammonium salt of formula (I) is quinclorac dimethylammonium.

5. The method according to claim 1 comprising as component B a solvent of formula IIa or a solvent of formula IIb.

6. The method according to claim 1 wherein the solvent is a solvent of formula IIa.

7. The method according to claim 1 wherein the solvent is a solvent of formula IIb.

8. The method according to claim 1 wherein B is a straight-chain or branched C₂-C₈-alkylene.

9. The method according to claim 1 wherein component C is selected from the group consisting of
c1) glyphosate; and
c2) bilanaphos (bialaphos), glufosinate and glufosinate-P;
including their agriculturally acceptable salts or derivatives.

10. The method according to claim 1 wherein component C is selected from the group consisting of
c1) EPSP synthase inhibitors: glyphosate, glyphosate-isopropylammonium, glyposate-potassium and glyphosate-trimesium (sulfosate); and
c2) glutamine synthase inhibitors: bilanaphos (bialaphos), bilanaphos-sodium, glufosinate, glufosinate-P, glufosinate-ammonium and glufosinate-P-ammonium.

11. The method according to claim 1 wherein component C is selected from the group consisting of glyphosate, glyphosate-isopropylammonium and glyphosate-trimesium (sulfosate).

12. The method according to claim 1, wherein component C is selected from the group consisting of glufosinate, glufosinate-P, glufosinate-ammonium and glufosinate-P-ammonium.
